SEQUENCE LISTING (18 sequences)

SEQ ID NO: 1 - protein, 1069 aa, synthetic construct
MGSSHHHHHHMGSVANSENVPYGKRLLPHIVDEVASKDPNRECIQIPWSSEPSDGWRTITWKDMANGINRCAHRIVELFGTPQEGSFPTIAYIGPNDARYVLMMIAASKAGYKALFISPRNSKEGQINLFKTTDCNILAFASSHKDTIKPWIAEHSMQTFEVSPMDSWFPDTEVPHFTYTKTFEDAEWDPLLVLHTSGSTGLPKPIVVKQGMWAGMDAYHNLPKFEGKNVAMREWAERAKRHFIPMPLFHIAGLLSVVNLSLYFDTPVALGIPDRPLSADNVIECLENLDVESAFLPPAVIEDMSQTEEGTKVLTSLNVVAFGGGNLAREAGNRLVKQGAVFMNLIGSTEFAPFPTYARNDPEAWQYFVYNPNVMGCEFRKQGDEDVYEMVVVRQNKHPGLQGIFYTFPELNEWSSRDLYRPHPTKPYHWIYHGRADNIIVFSNGEKLNPVTIEEIVSDHPSLKGALVVGSEKFQAGLIIEPYETPKNAAEEQALLDSVWPLVERANEETVAHGRISRDMIRLSNPQKPFLRAAKGTVQRAGTIKLYADEIEKLYEKESSDQDQDAPKLDISSEDALTASIVDALRKDVGADKLDADVDFFAAGIDSLGVMRAAKLLRAGLKEAGHEVDAKSFATRVIYQNSTPRRLASHILNAFINGQGESLSEDEQQIQAMEVIWNKYTSNLTKAVPNRPDPNTKDQTVILTGSTGMLGSYLLDFMGRNPRVSKIICFNRAADGGRAQGLKACVERGLDATALKTKAEFYQVDLSQPKLGLSDDVYARLQAETDRVIHNAWPVNFNIPIESFDPSLAGVRHIADLAATAAKRVAVTFIASIAVADRWQEDGKVPETRLEELALANGGYGRSKMVGSLILEDAAAKGAGDFPYAIVRVGQIAGPEGEAGEWNKHEWLPSIIASSLYLHALPKNLGTMDRVDWTPVERVAKLVLEASGTARVVQSADEINGYYHGVNPNYTQWYDLAVAVQEFYGKDKIPEFISFSEWVDRLEKSQADGPESVAANPGVKLIDTYRAMASADKAVVYDMQKTLERCPVVKETKAITPEMMKHWCAQWRH

SEQ ID NO: 2 - protein, 1071 aa, synthetic construct
MGSSHHHHHHMEAVVDTTSETAKPPQRLLATVVDSLALECPTRRFCLIPNGKDVHQGFREVTFRDLCCAVNRMSWWMEKHLASSVKGATIAYLGSNDIRYIILMLASHKTGCTIFFPSTRLSNEAYDSVFGATQTKMLLFSPEKHPLVSGLTGPSKAISSLEVPSVPEMLNDNPDVKNYPFTSTFEEFEDKTAFIIHSSGTTGMPKPVSLTHGYLGTVDYSAFMPRPAGRSPSFFQDLLSADPHPRDPVLSVTPYFHIMGLVSFFVSIFHNIPFVTISDQPLSVSLLVDIIRATHPTATILPPSILEDMSLSQEALECLGTLKFVCYGGAPLAKQVGDKVSQYTQLRNAIGSTEIGIIGSLVPEGKENWGYFEWNPAYGIDMQPVADDVYELVIPRLEDSRRMHGIFHTFPSFKEYRSKDLYVRHPKIPKLWQYRGRLDDVIVLSNGEKLNPVTLEKVVEGHPFVRRALVFGQGRFQTGLLIEPAMDDRAGKIDERNFVDMIWPLVQTANQNVPRYGQVLKNMIRLASPAKPFKLTPKGTTQRHAVNADYAEEIDAMYATHEKQLGPKLPSTIDSESVHCYVHEVITSLTGRSDIRPSDDLFGLGLDSLQATQLSNILRSAVLSYNPALSTENITVQNIYTRPTTDKLAGLLLGVLQEQKEQAAIAPTESRSERIAGLVSKYTADLPARYVNSPTQLPRLSTVILTGSTGSLGTYILSGLLNDPHVAKVYCFNRAADAATRQRQGFAEKGLDASLLEDPSKVEFLHVSFGDKHFGLDDSMYSKLLDTVDLIVHNAWKVNFNHPVSSFEDPHIKGVREFVNFSLEARYNTHLAFVSSVSTIAGWTPSSDESAVPELPMDTVDAVLKQGYGESKHVGERICLEASRTSGVPTSVLRVGQIAGPDSRLGLWNPHEWLPSVVKTSKSMGKVPDTLGSVLVDWIAVDTLAKITIEILLSRRSSLSTQRHAVFHLTNPSQIPWASLIPAIQERYPMSVVSLAEWVEELEHIRNPSPQDLAKRPALKLLSFYQALARNAGAPNAEISVENSKKASRTMASLGPVSLAQMSNWLNQWDF

SEQ ID NO: 3 - protein, 1103 aa, synthetic construct
MGSSHHHHHHMTVNGHHTNGVNGANGTNGHANGSNGINDTKAVKEIVPFVKPQVNFASAQRLEGCIHSLPELVDFNSLNNQHHTFCVQAKSSEPFDTITHGEFKVAVSKCAAWLKENLPIRPSSDDKALTKMAPVALFMESDIGLVIHEFALMSIGVPPLVLSPRLSPVAINALLEATGAASFIVSPRMSEPLKGALAALAAKGVSTHIGNPYKAYYQPGADPKSVAPFEVPQNPEDVILLLHSSGTTGLPKPIPTTHRQLLFAVNCHKFDTEEQAQSLNLSTLPLFHGFGLVAPGLSMSAGKPTLYPASDGIPNAKSIVDLINKTNAKSMMTVPFLLDDITNLPNEEGIKALVHMDFVGTGGAALGAGIGDRLAKGGVKLLNFYGTTETGPLSLTFAPTDNYDWKYFRLRTDCEYKIDELEPRDGERRFRLTVYPYGSEGFEISDQLIRNEQYPETDFAAVGRDDDVIVLATGEKANPLILETKLTEAPMVKAAIAFGENQFNLGVIVEPAEPLTPDTESAFRESIWPIITAACDQMDAFSRIPSPDAVVLVPAGVVIPRTDKGSIARKETYALFDKQIKGVYEQLLKAAADAVEPLDLDNLEQNLKSLIQEHLHIQAPASDWGVEDSLFDIGVDSLQVLQLRRILVTAASKTEAFKDTDCEKMIPPEFVYMNPSIREIAAALTKGSDGGDVSLEDAAKEVVELAETYSLKGVSAQEKAPSSSEGAFVMLTGATGSLGSHVAADLARRDNVAKVVCLVRKDKGTNQPPMPGGNPFDKKILKARGIQLTDEQFGKLATLEVDPTADKLGLIPMAYGMMQAKVTHVIHAAWPMNYLIRLRNFQYQFKFLRNLLEFASQGPAPTKKRFVFISSIATVARIGLAQPGSISEAPVSPSDSACGIGYADGKLVCEKIMEKAAQDYGGQLDVTSVRCGQMTGSKKTGVWNSNEQIPMLLKSAQGLGSLPQLSGELSWIPVDDAASTVSEIAFSDGSMPIVQHLENPIRQSWDAMLQSFGRELGLPAGKVPFGEWLDQVAAADGDDETFPVKKLTFFFKSFFQSVACGQVVLDTTVSRGQSKTLNAMTAVGDETVKAYADYWKSTGYLSK

SEQ ID NO: 4 - protein, 1147 aa, synthetic construct
MGSSHHHHHHMQNPELGLAEVVATVMDGYADRPAVGDRVTEKVTDPATGRTTLRLLPQFDTLTYREVWRHAGAIASAWQHDPDRPLRAGDFVCTLGFTSSDCATVDLAALRLGAVAVPLQAGASASALRPILAETAPKAVASSIELLDTVVDAVLAESAPPWLIVFDYHPDIDDQRERFEAAARRLAEAGAPTVLEPLAEVISRGETLGPAPLNVPAPDENPLSLLIYTSGSTGTPKGAMYTQSMVRGMWRGDPDDDRPAVTLNFMPMSHIAGRVALIGTLVRGGLCCFTASSDMSTLLEDYSLVRPTELMVVPRVCELVHERFRAEVDRRAIDSADDTAIEEAVRAELREQVFGGRVQVVMCGSAPLAPEIAEFMRTCLRVPVFNGYGATEAGGIVTINGQVQRPPVIDYKLADVPELGYYRTDSPHPRGELLIKTKQLFPGYYRRPEITAEMFDEDGFYRTGDIMAEVEPDHLEYVDRTKNVLKLSQGEFVTVAAVQSVLTQAPLVRQIFVYGNSERSYLLAVVVPTPDAVERNAGSEPALRAAIAESLRRRARDAGLASYEVPRDFLIETEPFTQANGLLSDVGKTLAPRLRERYGERLEQLYAELAAGQDAQLSELRRTGRDRPVLETIGKAAQALLGCAGTDVRPDAHFTDLGGDSLSALTISTLLEEIFGVPVPVGVVTSPATDLRALAAYVEAERASTATRPTFATVHGANATEVRATELTLDRFLDADTLAATRDLPPAAETANTVLLTGSTGYLGRFLCLEWMRRLDERGGRVIAVVRGRDAESARERLDAAFDSGDEELARRYAKLAADTLEVLPGDIGEPRFGLDEQTWNRLATEVDLIVHPAALVNHVLPYDQLFGPNVAGTAEVIRLALTSRLKPVTYLSTVGVAGQASASALDEDADIRATNPVRRVDDSYASGYGTSKWAGEVLLREAHDLCALPVAVFRSDMILAHSAYTGQLNLPDMFTRLLFSLAVTGIAPYSFYLSGAGEPRPRAHYDGLPVDFTAAAVTELGERATTGFATYNTVNPHDDGVSLDVVVDWLTEAGRPIERIDDYADWFVRLETALRGLPENRRQYTLLPLLHAYAAPDLPIPGSVIPATRFRAAVRSAGIGTDGDIPHLSAELIAKYVSDLTHLRVL

SEQ ID NO: 5 - protein, 1158 aa, synthetic construct
MGSSHHHHHHMAEPLDAAAVPAHDPGQGLAEVLASVEPGRALAEVMASVLESHGDRPALGERARDPETGRLLPHFDTISYRELWSRVRALAGRWHHDPAYPLGPGDRICTLGFTSTDYATLDLACIHLGAVPVPLQSNAALPRLAPIVEESGPTVLAASVDRLDTAVDVVLASRTIRRLLVFDDGPGTTRPSGALAAARERLAGSPVTVDTLAELIDRGRDLPPPPLHTPDPGEDPLALLIYTSGSTGAPKGAMYTQRLLGTAWYGFSYGAADTPAISVLYLPQSHLAGRYAVMGSLVKGGTGYFTAADDLSTLFEDIALVRPTELTMVPRLCDMLLQHYRSELERRSDEPGDIEAAVRKAVREDFLGGRVAKAFVGTAPLSAELTAFVESVLGFHLYTGYGSTEAGGVLLDTVVQRPPVTDYKLVDVPELGYYATDLPHPRGELLLKSHTLIPGYYRRPDLTATIFDADGYYRTGDVFAETGPDRLVYVDRTKDTLKLSQGEFVAVSRLETVLLDSPLVQHLYLYGNSERAYLLAVVVPTPAALAGSGGDTEALRPLLMESLRSVARRAGLNAYEIPRGILVEPEPFSAGNGLFTESHKLLRPRLKERYGPVLELLYDQLADGQDRRLRELRRTGADRPVPETVVRAAQALLGCLSSDLRPGAHFTDLGGDSLSAVSFSELLKEIFHVDVPVGVIIGPAADLAEVARYITAARRPTGIRRPTFASVHGEHLTEVRAGDLVPEKFLDAPTLAAAPGLPRPDGDVRTVLLTGATGYLGRFLCLEWLERLAPSGGRLICLVRGSDATVATRRLEAAFDSGDAALLRRYRKAAAKTLEVVAGDIGEPLLGLAEDTWRELAGTVDLIVHPAALVNHLLPYGELFGPNVVGTAEVIRLALTARLKPVNHVSTVAVCLGTPAETADENADIRATVPVRTIGQGYADGYATSKWAGEVLLREAHERYGLPVAVFRSDMVLAHRTYAGQVNVPDVLTRLLLSLVNTGIAPGSFYRTDTRAHYDGLPVDFTAEAVVALGARVTEGHRTFNVLNPHDDGVCLDTFVDWLIEAGHPIRRIDDHGAWLTRFTAALRALPEKHRQHSLLPLIGAWAEPDEGAPGPLLPAERFHAAVRAAGVGPERDIPRVSPDLIRKYVTDLRALGLLVDP

SEQ ID NO: 6 - protein, 1158 aa, synthetic construct
MGSSHHHHHHMAEPLDAATASAHDPGQGLAEALAAVEPGRALAEVMASVLEGHGDRPALGERAREPETGRLLPHFDTISYRELWSRVRALAGRWHHDPEYPLGPGDRICTLGFTSTDYATLDLACIHLGAVPVPLPSNAPLPRLAPVVEESGPTVLAASVDRLDTAIDVVLASSTIRRLLVFDDGPGATRPGGALAAARQRLSGSPVTVDTLAGLIDRGRDLPPPPLYIPDPGEDPLALLIYTSGSTGAPKGAMYTQRLLGTAWYGFSYGAADTPAISVLYLPQSHLAGRYAVMGSLVKGGTGYFTAADDLSTLFEDIALVRPTELTMVPRLCDMLLQHYRSERDRRADEPGDIEAAVTKAVREDFLGGRVAKAFVGTAPLSAELTAFVESVLGFHLYTGYGSTEAGGVLLDTVVQRPPVTDYKLVDVPELGYYATDLPHPRGELLLKSHTLIPGYYRRPDLTAAIFDADGYYRTGDVFAETGPDRLVYVDRTKDTLKLSQGEFVAVSRLETVLLDSPLVQHLYLYGNSERAYLLAVVVPTPDALAGCGGDTEALRPLLMESLRSVARRAGLNAYEIPRGILVEPEPFSPENGLFTESHKLLRPRLKERYGPALELLYDRLADGQDRRLRELRRTGADRPVQETVLRAAQALLGSPGSDLRPGAHFTDLGGDSLSAVSFSELMKEIFHVDVPVGAIIGPAADLAEVARYITAARRPAGAPRPTPASVHGEHRTEVRAGDLAPEKFLDAPTLAAAPALPRPDGDVRTVLLTGATGYLGRFLCLEWLERLAPSGGRLVCLVRGSDATVAARRLEAAFDSGDTALLRRYRKAAGKTLDVVAGDIGEPLLGLAEETWRELAGAVDLIVHPAALVNHLLPYGELFGPNVVGTAEAIRLALTTRLKPVNHVSTVAVCLGTPAETADENADIRAAVPVRTTGQGYADGYATSKWAGEVLLREAHERYGLPVAVFRSDMVLAHRTYTGQVNVPDVLTRLLLSLVATGIAPGSFYRTDTRAHYDGLPVDFTAEAVVALGAPITEGHRTFNVLNPHDDGVSLDTFVDWLIEAGHPIRRIDDHGAWLTRFTAALRALPEKQRQHSLLPLIGAWAEPGEGAPGPLLPARRFHAAVRAAGVGPERDIPRVSPDLIRKYVTDLRALGLLAGP

SEQ ID NO: 7 - protein, 1168 aa, synthetic construct
MGSSHHHHHHMTTGDEHSRGAGRLESVDAAVRDPTDGLAHSIAALMEAYAERPALGERAREAVTDPVSGRTALRLLPRFTTITYAELWERAGAVAAEWTLDDQRPVKPGDFVATYGFTSVDHTVLDLACLRLGAVAVPLQSGAPVSRLRPVIKETGPRVLAASVEVLDSAVELVLASASKPRLVVFDHHPEIDDEREKFEAARRRLADAGLSCIDALTAVTERGRTLGCPPVYRPAASDDDPTRLLIYTSGSTGTPKGAIYTERMLTRLWTGWLPAQDALSPTTINYMPLSHMAARASLYGTLGHGGTACFTAKSDMSTLFEDMSLTRPTQLLLVPRVCDMLHEEYRTESARRAPEFTDTDALETAVRADLRERRLGGRVRQVTCGTAPISPELKRFVESCLEVPLHNGYGSTEAGPVLLDSRVQRPPVIEYKLVDVPELGYFTTDVPYPRGELLLKTECVTPGYYKRPEATAAVFDEDGFYRSGDIMAETGPDQLVYVDRRNNVLKLAQGEFVTVSRLESVFVTSPLIRQIYVYGNSERAYLLAVIVPTDEAVRQATSHDDLRRTLAESLQLMARQAELETYEIPRDFLVEPEPFSVENGLLSEVRKNLRPQLRNRYGDGLEALYEQLAGGRQEVLRDLREAGPGQPVFEAIRRAASALLGSPAADLSPTARFTDLGIDSLSALSFSRLLHDIFEVEVPVGVLLSPANNLKGIADHIEAARVSGTRRPSFATVHGPGSSAARAADLTLEKFIDSETLAAALRRGRPPEQSAPRTVLLTGANGYLGRFMCLDWLERLATTGGRLVCIVRGRDNADARRRLDAAFDSGDEELLHRYRDLAARRLDVFAGDVGQGRLGLDQDMWKSLTEDIDLIFHPAALVNHVLPYDQLFGPNVAGTTELIRLALTGRTKPFTYLSTVGVATALDPSRLDEDADIREVSPVRELSDAYAGGYATSKWAGEVLLREAHDACGLPVTVLRSDMILAHRRHTGQLNVPDMFTRLLFSLVTTGIAPTSFYRTDDDGNPRRAHYDGLPVDFVARAVNTLGTDNTDGYRTYNVVNPHDDGISLDTFVDWLTDAGHTIHRIDDHGTWLTRFETALRALPETQRRYSALPLLHAFRRPDEPVNGSLVPAHRFEKAVQEAGLDGGGIPHLSTDLIAKYVTDLRHLNLL

SEQ ID NO: 8 - protein, 1188 aa, synthetic construct
MGSSHHHHHHMTETISTAAVPTTDLEEQVKRRIEQVVSNDPQLAALLPEDSVTEAVNEPDLPLVEVIRRLLEGYGDRPALGQRAFEFVTGDDGATVIALKPEYTTVSYRELWERAEAIAAAWHEQGIRDGDFVAQLGFTSTDFASLDVAGLRLGTVSVPLQTGASLQQRNAILEETRPAVFAASIEYLDAAVDSVLATPSVRLLSVFDYHAEVDSQREALEAVRARLESAGRTIVVEALAEALARGRDLPAAPLPSADPDALRLLIYTSGSTGTPKGAMYPQWLVANLWQKKWLTDDVIPSIGVNFMPMSHLAGRLTLMGTLSGGGTAYYIASSDLSTFFEDIALIRPSEVLFVPRVVEMVFQRFQAELDRSLAPGESNSEIAERIKVRIREQDFGGRVLSAGSGSAPLSPEMTEFMESLLQVPLRDGYGSTEAGGVWRDGVLQRPPVTDYKLVDVPELGYFTTDSPHPRGELRLKSETMFPGYYKRPETTADVFDDEGYYKTGDVVAELGPDHLKYLDRVKNVLKLAQGEFVAVSKLEAAYTGSPLVRQIFVYGNSERSFLLAVVVPTPEVLERYADSPDALKPLIQDSLQQVAKDAELQSYEIPRDFIVETVPFTVESGLLSDARKLLRPKLKDHYGERLEALYAELAESQNERLRQLAREAATRPVLETVTDAAAALLGASSSDLAPDVRFIDLGGDSLSALSYSELLRDIFEVDVPVGVINSVANDLAAIARHIEAQRTGAATQPTFASVHGKDATVITAGELTLDKFLDESLLKAAKDVQPATADVKTVLVTGGNGWLGRWLVLDWLERLAPNGGKVYALIRGADAEAARARLDAVYESGDPKLSAHYRQLAQQSLEVIAGDFGDQDLGLSQEVWQKLAKDVDLIVHSGALVNHVLPYSQLFGPNVAGTAEIIKLAISERLKPVTYLSTVGIADQIPVTEFEEDSDVRVMSAERQINDGYANGYGNSKWAGEVLLREAHDLAGLPVRVFRSDMILAHSDYHGQLNVTDVFTRSIQSLLLTGVAPASFYELDADGNRQRAHYDGVPGDFTAASITAIGGVNVVDGYRSFDVFNPHHDGVSMDTFVDWLIDAGYKIARIDDYDQWLARFELALKGLPEQQRQQSVLPLLKMYEKPQPAIDGSALPTAEFSRAVHEAKVGDSGEIPHVTKELILKYASDIQLLGLV

SEQ ID NO: 9 - protein, 1188 aa, synthetic construct
MGSSHHHHHHMGNTSQSNATRTQLEAHRKRRIAELLSAEPGLVDLKPLPEVFEATAERGLRLSEIIKRTLAGYADRPALGERAKEFVPVAGRTEMRLLPHFRTLTYSELWLRLSAVAADWYHHPEHPLRAGDFVAVLGFAGTDYTTVDLACSQVGAVAVPLHSNSTVAHIRPLVTEAAPRIFATSAERLEMVAEALTGDTSVRRLIVFDYHPELDEHREHMAAARRRLAEAGSPISLELLSHVIDCGRTSPPAPDLPQEDRAGETLSALIYTSGSTGSPKGVMYAESTVSLMWRPNWDPSYQFPVITANFFPQSHLVARRILATTLARGGTAYFVASSDLSTFLEDLALIRPTDLTVVPRVCEMLFQGYQSRLDQRAPRAELREADYTEVLAHFRDEVFGGRIVRILCGSAPLAPELVAFLESCLEVPFHNGYGTTETGFLMLDHRVQRPPVVDYKLVDVPELGYLTTDSPHPRGELLVKTRLITSGYYQRPELTAEVFDEEGFYRTGDIMAEIEPDNLVYIDRRNNVLKLSQGEFVALARLESVFATSPLVHQIYLYGNSSRAYLLAVVVPTHPAIQQASDRAALTSTISESLQRIAKEAKLNPYEIPRDLLIETEPFSQDNGLLSDSTKHIRPRLRERYGEQLERRYAELAEGQANELRGLRHDKSRPVVETVTRAVQALLGMPEGELNPSTRFTDLGGDSLSALSLSHLLRETFDAEVPVGTLIGPANSLRQIADHIEQLRSPSTERPTFTRVHGEHATAARAEDLTLDTFIDQDTLTAATSLPQPTDTPGTVLLTGANGYLGRFLCLEWLERLAPIGGTLICLVRGADNTAARRRLEQALDSGDAELQRRFGELATNGTLQVLAGDIGMANLGLTDQTWQQLAATVDLIVHPAALVNHVLPYRQLFGPNVVGTAELIRMALTTRLKPISYLSTVAVASHGAATLDEDIDIRVASPARPIDERHAGGYALSKWAGEVLLREAHDLCGLPVTVFRSDMILAHGRYTGQLNVPDTFTRLMLSLIATGIAPQSFYRTEDSGQQPRAHYDGLPGGFVASSVATLGDGTAGYRTFNVVNPHDDGISLDTVVDWLADSGTPIHRIEDYQEWHERFEAALRALPEKQRQHSLLPIVHAYREPATPLAGSVVPATRFQEAVRAAGIGSEKEIPHLSRPLIEKYLTDLRHLGLV

SEQ ID NO: 10 - protein, 1203 aa, synthetic construct
MGSSHHHHHHMHQLTVTGMNICEVQRLFPRMTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 11 - protein, 1898 aa, synthetic construct
MGSSHHHHHHMADSLPLLAPQDSYQSGLAACVSEREESILTRVAAKPEMPRVAYAMVFPEEPPFAPHPNDPDIEALLAGGGLEVLTKSFLLGRLQWTPLPCVCRPAEAGAARRTFDASLPSSALFGDGGAVFVRVAHMARRRWRHNWVAHFAASDSTAELAALHPHLPTISGGQRSHVSNEAMDTRATVALCEARGSHPDAWPAAGSPSRRWCCGLELPPRTRSVCVVVGVLDAERSVPLVRSTYVPGLQWPMQSSLAKEADSTESREAFQAGKPYWEAPIAPAPTDGSVASPTEIEPRPWYDFEVSSRSASADVGWPVFVGRVKVTIRREQPDGAPPIEGPVELEVIGASLANFRDFQTRRSLIPRSFWRAKLVIPSGQTSAFCLPRRCAQIVRVVLASPSPASLWVHDIDVRIASCWGSTPQDDMAASLDLFGPRPALGMRFPIAGGAAAAAGPAARVADPAAASPSPPSASQGAGKDDALSGVLEEAGTGSEDKLGPYEWLSYKDFATFANRTSSGLEGSLLGLLGDGDATRRLPPPPADSEAAGSAAAGSAAADSAAAGGSASSAGSGADSSDRVFMGICASNRMEWLAAELAGQHGSFVHVPIMTTASQEVVDHVVSQTGMMVAVAEPGEPLARLLSTQKRLGLPELLVVIDTTHFGARDPACPDVTADPAFAPAAQLVDGSARKAGRAFALARLSDIVEEGRRRLVAHGHESEAGERLLRFPMPTQRMRDDAKAAFAECSEERQTQLALLRHPDRIASPGWASSRAWVLTVDTSKPPGGAGRRVALGQGPGLSDLACVVYTSGSTGLPKGVQRSFQSNMEALHEFFAPTVAVHFSVQPLAHLSEAHSLPSVLVSGGQVGFATGGRHGVYGDVAELEPTFLNTVPAFFNRLHALFSAALAVKAAGAPEAARAAIRQALLKEFQVALGGRLQSIGIGSAPVTKGVLDWMTVCFSQAQVGEGYGSTECGTISNGNKIAEGVEFRLDDIPELGYLTSGSPPRGEILVRTKWSSEGYFRNPKATSDALTDDGFFRTGDVGEQLEDGRVMIIGRRKFVTKLANGEFVSLERIETVLSKSDLVDQVFVDADGSEYGVVAVVVAVPTALAAAVSARPAGKQDADALRKLARSAEAAAALKADLARVAAESGLQPFEAPKAVFLETELRFTAENGLLTGSNKTSRSGLRRKYSAIVKALYSVAGANPGADELLKRAAELGAAGGDSDGPGAAAAGGAAAAEPTSVEDGVRATVSRLVFETLGVSDGDLVAALGSDSLRVSSLSAMVGSALGVDLAAAANRASTVRELVDEVTALVLESRGLARAPSAAAGAGGKAPLHGAAETATADMEALGGWVASAMGGVPAWEAERGVDIALATDVPKPGSQSSIASAEAETVLLTGATGFLGVHLLVQLLQQSDKRVVALARPSRGTSGAQGIRQARVGAESEAEVDGGGSSAAAPARQTSAGRTGDELAEQRVVAAVKETRCDLPAGWRGRLTVLASDIAKPMLGLPAARWRELRDDPSLCVVHCAARVNWLMRYEQLRASNVLGTLEVIRLCAAGASRHPLHFVSTISVGDTARGAAESDRMPFDRVVAGLQAGQGGYGPSKWLAEAAVVRAGEHGTAGSGLFVSVHRPGMITAHSQTGHSNEQDFVNRYIAACASLGVALGPGLPADARLDMTPVDFVAGGVTRIALAHGAKASAPDAGWRRSGSCFQYVNADGSPRFADIGAWIREAGYNCEPRGYADFRDELHSRAAEGHALTPLLDFFPPGDFPESLGAVHGGDTLTRAALEDLAEAGTADPGDAALVPAPPKVTAVVIRRTLASLAVRGFVPAPDAAAPSVPETTLRPARQVLEAASREAAESHRGRTNREVLALLTQAVQDDAEVDLSPLLRSLVDAYG

SEQ ID NO: 12 - protein, 1183 aa, synthetic construct
MGSSHHHHHHMSTATHDERLDRRVHELIATDPQFAAAQPDPAITAALEQPGLRLPQIIRTVLDGYADRPALGQRVVEFVTDAKTGRTSAQLLPRFETITYGEVAQRVSALGRALSDDAVHPGDRVCVLGFNSVDYATIDMALGAIGAVSVPLQTSAAISSLQPIVAETEPTLIASSVNQLSDAVQLITGAEQAPTRLVVFDYHPQVDDQREAVQDAAARLSGTGVAVQTLAELLERGKDLPAVAEPPADEDSLALLIYTSGSTGAPKGAMYPQSNVGKMWRRGSKNWFGESAASITLNFMPMSHVMGRSILYGTLGNGGTAYFAARSDLSTLLEDLELVRPTELNFVPRIWETLYGEFQRQVERRLSEAGDAGERRAVEAEVLAEQRQYLLGGRFTFAMTGSAPISPELRNWVESLLEMHLMDGYGSTEAGMVLFDGEIQRPPVVDYKLVDVPDLGYFSTDRPHPRGELLLRTENMFPGYYKRAETTAGVFDEDGYYRTGDVFAEIAPDRLVYVDRRNNVLKLAQGEFVTLAKLEAVFGNSPLIRQIYVYGNSAQPYLLAVVVPTEEALASGDPETLKPKIADSLQQVAKEAGLQSYEVPRDFIIETTPFSLENGLLTGIRKLAWPKLKQHYGERLEQMYADLAAGQADELAELRRNGAQAPVLQTVSRAAGAMLGSAASDLSPDAHFTDLGGDSLSALTFGNLLREIFDVDVPVGVIVSPANDLAAIASYIEAERQGSKRPTFASVHGRDATVVRAADLTLDKFLDADTLASAPNLPKPATEVRTVLLTGATGFLGRYLALEWLERMDMVDGKVIALVRARSDEEARARLDKTFDSGDPKLLAHYQQLAADHLEVIAGDKGEANLGLRQDVWQRLADTVDVIVDPAALVNHVLPYSELFGPNALGTAELIRLALTSKQKPYTYVSTIGVGDQIEPGKFVENADIRQMSATRAINDSYANGYGNSKWAGEVLLREAHDLCGLPVAVFRCDMILADTTYAGQLNLPDMFTRLMLSLVATGIAPGSFYELDADGNRQRAHYDGLPVEFIAAAISTLGSQITDSDTGFQTYHVMNPYDDGIGLDEYVDWLVDAGYSIERIADYSEWLRRFETSLRALPDRQRQYSLLPLLHNYRTPEKPINGSIAPTDVFRAAVQEAKIGPDKDIPHVSPPVIVKYITDLQLLGLL

SEQ ID NO: 13 - protein, 1062 aa, synthetic construct
MGSSHHHHHHMRSFVKANVDFSSAERKEDYIHSLPELVDFNAVQNPNHLLCIQARSNAPWVKITNAQFKVAIDQCATWIAENVKLPKARTKHDLTGRLPVALLMESDFGLLVHQFALVSMGIPPLVLSARLSPEAIFHLLRSTEASSLIVSQRVAMITKGAFGNVKTSDFHVAQPYSTFCNVPADKSVRKQSVYPDNIDANIVLLHSSGTTGLPKPIALSHRQLMFSVSHGDFETEEEAQGIVISTLPLFHGFGLLAPGLSMAIGKTVCFPASDEVPDAQSIVDLINMSGATGMLTVPFLLENMAALPNGTGLRALAKLDFVGTGGSALSADFGVSASAAGVKLLNLYGTTETGPLTKTFAPKSGYDWKYFRLRQDMLFKVTELPPVDGEKRFRLTVFPFGADKPFEIADQLIRSEKFPETDFAAVGRDDDVVVLATGEKVNPLLLETALTDSGLVKSAIVFGENQFQIGVVVEPATPLNPDQKEEFRKKIWPIIVRVGERMDTTARIYSPNAVIVVPSSVTIPRTDKGSIARKEVFQLLEKEISQVYEDLENGSIEETPLDYDKLEQELKGLIQKRLKLRVHPGKWTVDDNLFHLGLDSLQATTLRRILLSAASKTPPDVIGKDFIYVNPSVKAIANALRPANGPIGTESASVAQEVDDYAQQYSIKGFEVQDIVPKASPKLIRGAVVLLTGSSGGLGSHALGKLAESTQVAKIVCLQRKRPGTVINPIPGAAKVDRASIEAKGIKLTDDQWAKITALEIDPTIDNLGLPAMVMGMVSKTVTHILHAAWPMDFHMRLPSFGYQFSYLKNLLRIAVQAPQKVRFLFVSSISALAKLGLITPGRPIPEEPLDVESAACGIGYADAKLVCEKILEEAASLYNSNVEVVIARCGQLSGARKTGAWNVSEQIPMLIRTSQGLGILPILEGTVSWIPVDDAAATVAELLFAPDAPGLVTHVENPVRQSWSEVFQIIGNELRITKTLSFDDWLGEVTSTAERDVEDYPVRKLYEFFKLYFRIASSGAVVMGTDMSRKNSATLRCLKALDRGTIAGYVRYWRSVGYLRQ

SEQ ID NO: 14 - protein, 1062 aa, synthetic construct
MGSSHHHHHHMSQQQNPPYGRRLILDIIKERALNEPNREWVSVPRSSDPKDGWKILTYLDAYNGINRVAHKLTQVCGAAAPGSFPTVAYIGPNDVRYLVFALGAVKAGYKALFISTRNSAEAQVNLFELTNCNVLVFDQSYKATVQPWLHEREMTAILALPADEWFPADQEDFPYNKTFEEAEWDPLMVLHTSGSTGFPKPIVARQGMLAVADQFHNLPPREDGKLMWIVEMSKRAKRLMHPMPLFHAAGMYISMLMIHYWDTPGALGIGERPLSSDLVLDYIEYADVEGMILPPAILEELSRDEKAIQSLQKLNFVSFGGGNLAPEAGDRLVENNVTLCNLISATEFTPFPFYWQYDQKLWRYFNFDTDLFGIDWRLHDGESTYEQVIVRKDKHPGLQGFFYTFPDSSEYSTKDLYKRHPTHEDFWIYQGRADNIIVFSNGEKLNPITIEETLQGHPKVMGAVVVGTNRFQPALIIEPVEHPETEEGRKALLDEIWPTVVRVNKETVAHGQIGRQYMALSTPGKPFLRAGKGTVLRPGTINMYKAEIDKIYEDAEKGVATDEVPKLDLSSSDALIVSIEKLFETSLNAPKLEADTDFFTAGVDSMQVITASRLIRAGLAAAGVNIEASALATRVIYGNPTPKRLADYLLSIVNKDSNQGTLDNEHHVMEALVEKYTRDLPTPKQNKPAPADEGQVVVITGTTGGIGSYLIDICSSSSRVSKIICLNRSEDGKARQTASSSGRGLSTDFSKCEFYHADMSRADLGLGPEVYSRLLSEVDRVIHNQWPVNFNIAVESFEPHIRGCRNLVDFSYKADKNVPIVFVSSIGTVDRWHDEDRIVPEASLDDLSLAAGGYGQSKLVSSLIFDKAAEVSGVPTEVVRVGQVAGPSSEKGYWNKQEWLPSIVASSAYLGVLPDSLGQMTTIDWTPIEAIAKLLLEVSGVIDNVPLDKINGYFHGVNPERTSWSALAPAVQEYYGDRIQKIVPLDEWLEALEKSQEKAEDVTRNPGIKLIDTYRTWSEGYKKGTKFVPLDMTRTKEYSKTMREMHAVTPELMKNWCRQWNF

SEQ ID NO: 15 - protein, 1196 aa, synthetic construct
MGSSHHHHHHMTQSHTQGPQASAAHSRLARRAAELLATDPQAAATLPDPEVVRQATRPGLRLAERVDAILSGYADRPALGQRSFQTVKDPITGRSSVELLPTFDTITYRELRERATAIASDLAHHPQAPAKPGDFLASIGFISVDYVAIDIAGVFAGLTAVPLQTGATLATLTAITAETAPTLFAASIEHLPTAVDAVLATPSVRRLLVFDYRAGSDEDREAVEAAKRKIADAGSSVLVDVLDEVIARGKSAPKAPLPPATDAGDDSLSLLIYTSGSTGTPKGAMYPERNVAHFWGGVWAAAFDEDAAPPVPAINITFLPLSHVASRLSLMPTLARGGLMHFVAKSDLSTLFEDLKLARPTNLFLVPRVVEMLYQHYQSELDRRGVQDGTREAEAVKDDLRTGLLGGRILTAGFGSAPLSAELAGFIESLLQIHLVDGYGSTEAGPVWRDGYLVKPPVTDYKLIDVPELGYFSTDSPHPRGELAIKTQTILPGYYKRPETTAEVFDEDGFYLTGDVVAQIGPEQFAYVDRRKNVLKLSQGEFVTLAKLEAAYSSSPLVRQLFVYGSSERSYLLAVIVPTPDALKKFGVGEAAKAALGESLQKIARDEGLQSYEVPRDFIIETDPFTVENGLLSDARKSLRPKLKEHYGERLEAMYKELADGQANELRDIRRGVQQRPTLETVRRAAAAMLGASAAEIKPDAHFTDLGGDSLSALTFSNFLHDLFEVDVPVGVIVSAANTLGSVAEHIDAQLAGGRARPTFATVHGKGSTTIKASDLTLDKFIDEQTLEAAKHLPKPADPPRTVLLTGANGWLGRFLALEWLERLAPAGGKLITIVRGKDAAQAKARLDAAYESGDPKLAGHYQDLAATTLEVLAGDFSEPRLGLDEATWNRLADEVDFISHPGALVNHVLPYNQLFGPNVAGVAEIIKLAITTRIKPVTYLSTVAVAAGVEPSALDEDGDIRTVSAERSVDEGYANGYGNSKWGGEVLLREAHDRTGLPVRVFRSDMILAHQKYTGQVNATDQFTRLVQSLLATGLAPKSFYELDAQGNRQRAHYDGIPVDFTAESITTLGGDGLEGYRSYNVFNPHRDGVGLDEFVDWLIEAGHPITRIDDYDQWLSRFETSLRGLPESKRQASVLPLLHAFARPGPAVDGSPFRNTVFRTDVQKAKIGAEHDIPHLGKALVLKYADDIKQLGLL

SEQ ID NO: 16 - protein, 1184 aa, synthetic construct
MGSSHHHHHHMAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 17 - protein, 1184 aa, synthetic construct
MGSSHHHHHHMSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 18 - protein, 475 aa, synthetic construct
MGSSHHHHHHSQDPNSMQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

(19) United States
(12) Patent Application Publication
(10) Pub. No.: US 2025/0297292 A1
(43) Pub. Date: Sep. 25, 2025

(54) ENZYMATIC REDUCTION OR REDUCTIVE AMINATION OF ARYL AND HETEROCYCLIC CARBOXYLIC ACIDS AND ALDEHYDES

(71) Applicants: Aditya Kunjapur, Newark, DE (US); Madan Gopal, Newark, DE (US); Roman Dickey, Newark, DE (US); Wilfred Chen, Newark, DE (US); Priyanka Nain, Newark, DE (US)

(72) Inventors: Aditya Kunjapur, Newark, DE (US); Madan Gopal, Newark, DE (US); Roman Dickey, Newark, DE (US); Wilfred Chen, Newark, DE (US); Priyanka Nain, Newark, DE (US)

(73) Assignee: University of Delaware Newark, Newark, DE (US)

(21) Appl. No.: 18/862,430

(22) PCT Filed: May 2, 2023
(86) PCT No.: PCT/US2023/066492

§ 371 Date: Nov. 1, 2024

Related U.S. Application Data
(60) Provisional application No. 63/337,347, filed on May 2, 2022.

(51) Int. Cl. C12P 13/00

(52) U.S. Cl. CPC C12P 13/001

(57) ABSTRACT

The invention provides a method for preparing an amine product in a single reaction mixture, comprising incubating a biomass-derived or plastics-derived aryl or heterocyclic carboxylic acid, a carboxylic acid reductase (CAR), and a ω-transaminase (TA) in the single reaction mixture, reducing the biomass-derived or plastics-derived aryl or heterocyclic carboxylic acid to an aryl or heterocyclic aldehyde, and transferring an amine to the aryl or heterocyclic aldehyde. Also provided is method for reducing a substrate, comprising incubating the substrate and a CAR in a reduction mixture to produce a reduction product. Further provided is a method for transferring an amine to an aldehyde, comprising incubating the aldehyde and an amine transferring enzyme (e.g., ω-TA) in an amine transferring mixture to produce an amine product.

### CROSS-REFERENCE TO RELATED APPLICATION

This application claims priority to U.S. Provisional Application No. 63/337,347, filed May 2, 2022, and the contents of which are incorporated herein by reference in their entireties for all purposes.

### REFERENCE TO U.S. GOVERNMENT SUPPORT

This invention was made with government support under Grant No. EFRC DE-SC0021166 from the Department of Energy, Contract No. DE-AC02-05CH11231 from the Department of Energy, and Grant No. CMMI-1934887 from the National Science Foundation. The United States has certain rights in the invention.

### FIELD OF THE INVENTION

This invention relates generally to reductive amination cascades to act upon functionalized derivatives of lignin deconstruction products or plastic deconstruction products.

### BACKGROUND OF THE INVENTION

Aryl and heterocyclic carboxylic acids and aldehydes can be derived from waste sources such as biomass. For example, lignin is the largest natural source of aryl compounds and could be the best alternative to petroleum-based resources, and yet 98% is burned as waste each year. Although a barrier to its utilization is its complexity, technical advances made over the last few decades have led to the design of chemical and biological deconstruction processes that tend to funnel lignin toward a limited range of building blocks. Some of these building blocks have been functionalized with methacrylate chemistries and subsequently used to form materials such as pressure-sensitive adhesives. This approach of harnessing lignin as a source of polymeric materials could have broad utility if the building blocks could be chemically diversified to include sidechain chemistries that bestow useful properties to the polymer. Amines represent one attractive target due to their ability to react with aldehydes to form reversible imine bonds which can be utilized in complex epoxy networks or thermosets. Additionally, amine monomers fulfill growing orders in the automotive, aerospace, construction, and health industries. However, because neither lignin nor its deconstruction products contain nitrogen heteroatoms and instead contain an array of oxygen heteroatoms, a selective approach to the installation of amines on breakdown products is needed. Ideally, this approach would also remain compatible with downstream strategies for polymerization, such as the functionalization of acrylate-based block polymers.

The future of plastics production will require greater circularity to decrease dependence on petroleum as a raw material and to stem the ever-growing flow of plastic waste into landfills. Mechanical recycling of plastics has been the predominant technology to enable the reuse of postconsumer plastic waste. However, chemical deconstruction by synthetic or enzymatic approaches has emerged to allow for recovery of the monomer building blocks of various polymer plastics. While chemical deconstruction offers a simple path to remake the original, virgin-like polymer, broader adoption and utility of chemical deconstruction will benefit from the design of sustainable methods to add value to deconstruction products, making them amenable to upcycled applications. One of the most widely used single-use plastics is polyethylene terephthalate (PET), a member of the thermoplastic category of polymers. Chemical and enzymatic deconstruction methods have been applied to polyethylene terephthalate (PET) to produce products such as bis-(2-hydroxyethyl) terephthalate (BHET), mono-(2-hydroxyethyl) terephthalic acid (MHET), and terephthalic acid (TPA). To date, most efforts have harnessed these compounds for either the resynthesis of PET, biological degradation as catabolized carbon sources, or aliphatic building blocks derived from aryl ring cleavage.

Biocatalysis presents an industrially established and green alternative for synthesis of amines and diamines from carbonyl-containing precursors. Pyridoxal 5′-phosphate-(PLP)-dependent ω-transaminases (ω-TA, EC 2.6.1.x) have been previously applied for the conversion of aldehydes to amines using simple co-substrates like isopropylamine (iPr-NH₂), though to our knowledge, these enzymes have not been reported to have activity on bifunctional aryl aldehydes. CARs are multidomain and polyspecific enzymes that perform the desirable 2e⁻ reduction of carboxylic acids to aldehydes at a cost of adenosine 5′-triphosphate (ATP) and nicotinamide adenine dinucleotide phosphate (NADPH). Given the broad substrate scope of both CAR and ω-TA families, some precedent for this type of cascade exists for certain aliphatic or heterocyclic chemistries. In 2019, Fedorchuk et al. designed a CAR and ω-TA cascade for one-pot transformation of the dicarboxylate adipic acid to hexamethylenediamine, a diamine precursor to nylon (J Am Chem Soc 2020, 142 (2), 1038-1048). That study achieved only 30% yield, even after significant engineering to develop two CAR variants for use alongside two distinct transaminase orthologs. A major challenge was that each unique CAR or TA variant could accept a mono-functionalized intermediate or a bifunctionalized intermediate but not both.

There remains a need for a method for conversion of biological degradation products or oxidative deconstruction products to building blocks useful for making valuable polymers or therapeutic compounds.

### SUMMARY OF THE INVENTION

The present invention relates to reduction of a biomass-derived or plastic-derived aryl or heterocyclic carboxylic acid to an aryl or heterocyclic aldehyde by a carboxylic acid reductase (CAR), transferring of an amine to the aryl or heterocyclic aldehyde to make an amine product by an amine transferring enzyme, for example, a ω-transaminase (TA), and a single reaction involving the reduction by the CAR and the amine transferring by the ω-TA.

The present invention provides a method for preparing an amine product in a single reaction mixture. This single reaction preparation method comprises incubating a biomass-derived or plastic-derived aryl or heterocyclic carboxylic acid, a carboxylic acid reductase (CAR), and a ω-transaminase (TA) in the single reaction mixture, reducing the biomass-derived or plastic-derived aryl or heterocyclic carboxylic acid to an aryl or heterocyclic aldehyde, and transferring an amine to the aryl or heterocyclic aldehyde, whereby the amine product is produced in the single reaction mixture.

The biomass-derived aryl carboxylic acid may be a guaiacol and syringol 4-substituted carboxylic acid of Formula I

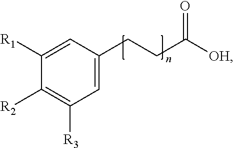

the aryl aldehyde may be of Formula II

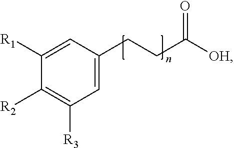

and the amine product may be of Formula III

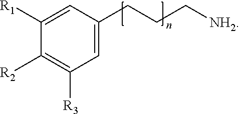

R₁, R₂, R₃ and n in Formula I, Formula II and Formula III may be selected from the group consisting of: (a) R₁═H, R₂═OH, R₃═OCH₃, and n=0; (b) R₁═OCH₃, R₂═OH, R₃═OCH₃, and n=0; (c) R₁═H, R₂═H, R₃═H, and n=1 (unsaturated); (d) R₁ ═H, R₂═OH, R₃═OCH₃, and n=1 (unsaturated); and (e) R₁═H, R₂═OH, R₃═OCH₃, and n=1 (saturated). R₁, R₂, R₃ and n in Formula I, Formula II and Formula III may be selected from the group consisting of: (a) R₁═OCH₃, R₂═OH, R₃═OCH₃, and n=0; (b) R₁═H, R₂═OH, R₃═OCH₃, and n=1 (unsaturated); and (c) R₁═H, R₂═OH, R₃═OCH₃, and n=1 (saturated).

The biomass-derived aryl carboxylic acid may be of Formula IV

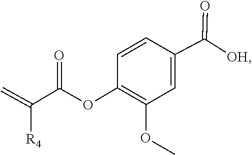

the aryl aldehyde may be of Formula V

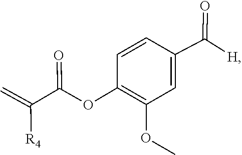

and the amine product may be of Formula VI

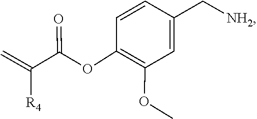

R₄ in Formula IV, Formula V and Formula VI may be H or CH₃.

The biomass-derived aryl carboxylic acid may be a furan carboxylic acid.

The plastic-derived aryl carboxylic acid may be a polyethylene terephthalate (PET)-derived aryl carboxylic acid. The PET-derived aryl carboxylic acid may be selected from the group consisting of terephthalic acid (TPA), mono-(2-hydroxyethyl)-terephthalic acid (MHET) and monomethyl terephthalate (mmTPA).

According to the single reaction preparation method, the PET-derived aryl carboxylic acid may be terephthalic acid (TPA) and the amine product may be para-xylylenediamine (pXYL). The single reaction preparation method may further comprise producing an intermediate selected from the group consisting of 4FBA, TPAL, pAMBA and pAMB. The single reaction preparation method may further comprise converting the TPA to 4FBA, converting the 4FBA to TPAL, converting the TPAL to pAMB, and converting the pAMB to the pXYL. The single reaction preparation method may further comprise converting the TPA to 4FBA, converting the 4FBA to pAMBA, converting the pAMBA to pAMB, and converting the pAMB to the pXYL. The pXYL may have a molar yield of 30-50%.

According to the single reaction preparation method, the PET-derived aryl carboxylic acid may be mono-(2-hydroxyethyl)-terephthalic acid (MHET) and the amine product may be para-(aminomethyl)benzoic acid (pAMBA). The single reaction preparation method may further comprise producing mono-(2-hydroxyethyl)-para-(aminomethyl)benzoic acid (MHE-pAMBA) as an intermediate. The single reaction preparation method may further comprise converting the MHET to mono-(2-hydroxyethyl)-para-(aminomethyl)benzoic acid (MHE-pAMBA), and converting the MHE-pAMBA to the pAMBA.

According to the single reaction preparation method, the ω-TA may consist of an amino acid sequence having at least 80% identity to the amino acid sequence of SEQ ID NO: 18. The ω-TA may be cvTA.

According to the single reaction preparation method, the CAR may be selected from the group consisting of an amino acid sequence having at least 80% identity to an amino acid sequence selected from the group consisting of SEQ ID NOs: 1-17. The CAR may be selected from the group consisting of an amino acid sequence having at least 80% identity to an amino acid sequence selected from the group consisting of SEQ ID NOs: 1-16. The CAR may be selected from the group consisting of srCAR, trCAR, msCAR, mavCAR, ncCAR, afCAR and niCAR.

According to the single reaction preparation method, the PET-derived aryl carboxylic acid may be TPA, the amine product may be para-xylylenediamine (pXYL), and the CAR may be selected from the group consisting of srCAR, trCAR, msCAR, mavCAR, ncCAR and afCAR, and the ω-TA may be cvTA.

According to the single reaction preparation method, the PET-derived aryl carboxylic acid may be mmTPA, the CAR may be srCAR or niCAR, and the ω-TA may be CVTA.

According to the single reaction preparation method, the PET-derived aryl carboxylic acid may be MHET, the amine product may be para-(aminomethyl)benzoic acid (pAMBA), and the CAR may be selected from the group consisting of niCAR, srCAR and trCAR, and the ω-TA may be cvTA.

According to the single reaction preparation method, the CAR may be expressed by first recombinant cells. The CAR may be purified from the first recombinant cells. The single reaction mixture may comprise the first recombinant cells. The ω-TA may be expressed by second recombinant cells. The ω-TA may be purified from the second recombinant cells. The single reaction mixture may comprise the second recombinant cells. The first recombinant cells and the second recombinant cells may be the same or different.

The present invention also provides a method for reducing a substrate. This reduction method comprises incubating the substrate and a carboxylic acid reductase (CAR) in a reduction mixture to produce a reduction product, wherein the substrate is selected from the group consisting of terephthalic acid (TPA), 4-formylbenzoic acid (4FBA), mono-(2-hydroxyethyl)-terephthalic acid (MHET), monomethyl terephthalate (mmTPA), and para-(aminomethyl)benzoic acid (pAMBA), and wherein the CAR consists of an amino acid sequence having at least 80% identity to an amino acid sequence selected from the group consisting of SEQ ID NOs: 1-17.

According to the reduction method, the CAR may consist of an amino acid sequence selected from the group consisting of SEQ ID NOs: 1-17. The CAR may consist of an amino acid sequence having at least 80% identity to an amino acid sequence selected from the group consisting of SEQ ID NOs: 1-16. The CAR may consist of an amino acid sequence selected from the group consisting of SEQ ID NOS: 1-16. The CAR may be selected from the group consisting of srCAR, trCAR, msCAR, mavCAR, ncCAR, afCAR and niCAR. The CAR may be srCAR. The CAR may be trCAR.

According to the reduction method, the substrate may be TPA and the reduction product may be 4FBA or terephthalaldehyde (TPAL). The CAR may be selected from the group consisting of srCAR, trCAR, msCAR, mavCAR, ncCAR and afCAR.

According to the reduction method, the substrate may be mmTPA and the reduction product may be para-(aminomethyl)benzoic acid (pAMBA). The CAR may be srCAR or niCAR.

According to the reduction method, the substrate may be MHET and the reduction product may be 2-hydroxyethyl 4-formylbenzoate (MHE-4FBA). The CAR may be selected from the group consisting of niCAR, srCAR and trCAR.

According to the reduction method, the substrate may be 4FBA and the reduction product may be terephthalaldehyde (TPAL).

According to the reduction method, the substrate may be pAMBA and the reduction product may be para-aminomethylbenzaldehyde (pAMB).

According to the reduction method, the CAR may be expressed by first recombinant cells. The CAR may be purified from the first recombinant cells. The reduction mixture may comprise the first recombinant cells.

The present invention further provides a method for transferring an amine to an aldehyde. This amine transferring method comprises incubating the aldehyde and an amine transferring enzyme in an amine transferring mixture to produce an amine product, wherein the amine transferring enzyme is selected from the group consisting of a ω-transaminase (TA), an amine transaminase and a reductive aminase. The ω-TA may consist of an amino acid sequence having at least 80% identity to the amino acid sequence SEQ ID NO: 18. The aldehyde may be 4-formylbenzoic acid (4FBA) and the amine product may be para-(aminomethyl)benzoic acid (pAMBA). The aldehyde may be terephthalaldehyde (TPAL) and the amine product may be para-aminomethylbenzaldehyde (pAMB). The aldehyde may be para-aminomethylbenzaldehyde (pAMB) and the amine product may be para-xylylenediamine (pXYL). The aldehyde may be terephthalaldehyde (TPAL) and the amine product may be para-xylylenediamine (pXYL). The aldehyde may be methyl 4-formylbenzoate (mm4FBA) and the amine product may be methyl 4-(aminomethyl)benzoate (mm-pAMBA). The aldehyde may be 2-hydroxyethyl 4-formylbenzoate (MHE-4FBA) and the amine product may be mono-(2-hydroxyethyl)-para-(aminomethyl)benzoic acid (MHE-pAMBA). The aldehyde may be a furan aldehyde. The ω-TA may be expressed by second recombinant cells. The ω-TA may be purified from the second recombinant cells. The amine transferring mixture may comprise the second recombinant cells.

### BRIEF DESCRIPTION OF THE DRAWINGS

FIGS. 1A-C illustrate target lignin-derivable chemistries and systems.

FIGS. 2A-B illustrate an envisioned reaction schematic showing a route to repurpose lignin-derivable small molecules in panel A to amine-containing monomers and subsequently polymers in panel B.

FIGS. 3A-B show evaluation of CAR and ω-TA specificity on lignin-derivable small molecules and demonstration of efficient conversion of carboxylic acid and aldehyde functional group to amines.

FIGS. 4A-D show HPLC chromatogram traces of whole-cell, one-pot bioconversions of lignin-derivable carboxylic acids to heterocyclic primary amines.

FIGS. 5A-G show a design of one-pot enzyme cascade strategies for the bioconversion of acrylated and methacrylated compounds to amines.

FIGS. 6A-C show strategies for valorizing furans derived from deconstruction products of PEF or lignocellulosic biomass.

FIGS. 7A-C illustrate an envisioned reaction schematic showing routes to mono- and di-amines from various breakdown products of polyethylene terephthalate (PET) plastic.

FIGS. 8A-C show the ability of an amine transferring enzyme to convert PET-derived aldehydes to their corresponding amines.

FIGS. 9A-F show the ability of several naturally occurring CAR enzymes to convert PET-derived carboxylic acids to their associated aldehydes.

FIGS. 10A-C demonstrate the rate of reduction of PET-derived carboxylic acids to their associated aldehydes or the ability of a lipase enzyme to cleave an ester group.

FIGS. 11A-E show selective one-pot enzyme cascades that are reduced to practice for synthesis of mono- or di-amines from PET-derived carboxylic acids.

### DETAILED DESCRIPTION OF THE INVENTION

The present invention relates to methods for reducing an aryl or heterocyclic carboxylic acid derived from either plastic or biomass by a carboxylic acid reductase (CAR), transferring an amine to an aldehyde by an amine transferring enzyme (e.g., ω-transaminase (TA)), and preparing an amine product from an aryl or heterocyclic carboxylic acid derived from either plastic or biomass with the CAR and the amine transferring enzyme in a single reaction mixture. The present invention is based on inventors' discovery of conversion of many of the products of biological or oxidative deconstruction, which form aryl carboxylic acids or aldehydes, to their corresponding amines by constructing a one-pot biocatalytic cascade. The inventors have shown the potential of reductive amination cascades to act upon functionalized derivatives of lignin deconstruction products and plastic deconstruction products. The inventors have demonstrated that these cascades function either as purified enzymes or in cells.

The inventors have produced useful mono-amine and diamine building blocks from known PET deconstruction products by one-pot biocatalytic transformations by taking advantages of substrate specificity of an ω-transaminase and diverse carboxylic acid reductases (CAR) towards PET deconstruction products. The inventors have first established that an ω-transaminase from Chromobacterium violaceum (cvTA) (Table 1, SEQ ID NO: 18) can efficiently catalyze amine transfer to potential PET-derived aldehydes to form the mono-amine para-(aminomethyl)benzoic acid (pAMBA) or the diamine para-xylylenediamine (pXYL); and then identified CAR orthologs that could perform the bifunctional reduction of TPA to terephthalaldehyde (TPAL) or the reduction of mono-(2-hydroxyethyl) terephthalic acid (MHET) to its corresponding aldehyde. After characterizing 17 CARs (Table 1, SEQ ID NOS: 1-17) in vitro, the inventors have shown that the CAR from Segniliparus rotundus (srCAR) had the highest observed activity on TPA. Given these newly elucidated substrate specificity results, the inventors have designed modular enzyme cascades based on coupling srCAR and cvTA in one-pot with enzymatic co-factor regeneration. When TPA was supplied, the inventors have achieved a 69±1% molar yield of pXYL, which is useful as a building block for polymeric materials. When MHET was supplied and subsequent base-catalyzed ester hydrolysis was performed, the inventors achieved a 70±8% molar yield of pAMBA, which is useful for therapeutic applications and as a pharmaceutical building block. The present invention expands the breadth of products derived from PET deconstruction and lays the groundwork for eventual valorization of waste PET to higher-value chemicals and materials. The inventors have discovered an enzyme cascade that converts TPA to pXYL or MHET to pAMBA in one pot, respectively. The inventors' retrobiosynthetic design focused on steps of aldehyde consumption and aldehyde generation.

The inventors have designed a route to convert PET deconstruction products to upcycled amines as alternatives to these products, while preserving the aryl nature of the terephthalate monomer. Diamines such as para-xylylenediamine (pXYL) are value-added monomers for both thermoset and thermoplastic polymers. pXYL can be a component of polyamides, polyimides, or non-isocyanate polyurethanes. Such materials would substantially expand the breadth of products derived from PET deconstruction if an environmentally friendly option were available for valorization. However, chemical synthesis of pXYL requires multiple steps, elevated temperatures and pressures, and strong organic solvents. While diamines can be used as value-added monomers, a significant opportunity exists for valorization of PET-derived monomers to mono-amines such as para-(aminomethyl)benzoic acid (pAMBA), which is an antifibrinolytic drug used to promote blood clotting and treat fibrotic skin conditions. Monofunctional molecules are often challenging to make from bifunctional substrates (e.g., TPA), resulting in poor atom economy and low selectivity. However, enzymatic PET deconstruction offers a previously untapped potential to leverage substrates like MHET, a unique carboxylate with a ester “protecting” group, which could allow for monofunctionalization of terephthalate.

The inventors have designed enzyme cascades featuring a single ω-TA and CAR variants to produce amines at high selectivity and yield in a one pot reaction from PET-derived monomers. The ω-TA from Chromobacterium violaceum (cvTA) was found to successfully accept several PET-derived aldehydes while harnessing iPr-NH₂ as an amine donor. Novel putative CARs with activity on TPA were discovered and showed unexpected substrate specificity, based on which the inventors have designed highly selective routes to pXYL or to pAMBA by coupling the CAR from Segniliparus rotundus (srCAR) to cvTA. The present invention enables green conversion of PET deconstruction products to aryl (di)aldehydes and (di)amines that could serve as platform intermediates for value-added polymeric materials or pharmaceuticals, and valorization of plastic deconstruction streams.

The term “biomass-derived aryl or heterocyclic carboxylic acid” used herein refers to organic molecules that contain an aryl or heterocyclic group and a carboxylic acid functional group and are derived from renewable biomass sources such as plants, algae, and waste materials. Examples of the biomass-derived aryl or heterocyclic carboxylic acid include vanillic acid, acrylate vanillic acid, methacrylate vanillic acid, syringic acid, trans-cinnamic acid, 4-Hydroxy-3-methoxycinnamic acid, and 3-(4-Hydroxy-3-methoxyphenyl) propionic acid, 2-furoic acid, and furan dicarboxylic acid.

The term “plastic-derived aryl or heterocyclic carboxylic acid” used herein refers to organic molecules that contain an aryl or heterocyclic group and a carboxylic acid functional group and are derived from plastic materials. Examples of the plastic-derived aryl or heterocyclic carboxylic acid include polyethylene terephthalate (PET)-derived aryl or heterocyclic carboxylic acid.

The term “polyethylene terephthalate (PET)-derived aryl carboxylic acid” used herein refers to aryl carboxylic acid products of biological or chemical deconstruction of polyethylene terephthalate (PET). Examples of the PET-derived aryl carboxylic acid include Terephthalic acid, 4-formylbenzoic acid, monomethyl terephthalate, and MHET.

The term “amine product” used herein refers to a product having an amine group.

The term “aldehyde product” used herein refers to a product having an aldehyde group.

The term “aryl aldehyde” used herein refers to an aldehyde having an aryl group.

The term “heterocyclic aldehyde” used herein refers to an aldehyde having a heterocyclic group.

The term “carboxylic acid reductase (CAR)” used herein refers to an enzyme that catalyzes reduction of a carboxylic acid to an aldehyde.

The term “ω-transaminase (TA)” used herein refers to an enzyme that catalyzes the transfer of an amino group from a primary amino donor to a carbonyl acceptor with pyridoxal 5′-phosphate (PLP) as a catalytic cofactor produce an amine product.

The terms “single reaction mixture” and “one pot” are used herein interchangeably and refer to a mixture of reagents under conditions suitable for carrying out two or more reactions.

The term “yield” as used herein refers to a percentage of a product formed by a substrate in a reaction based on the molar or weight amount the substrate as supplied to a reaction.

The present invention provides a method for preparing an amine product in a single reaction mixture. This single-reaction preparation method comprises incubating a biomass-derived or plastic-derived aryl or heterocyclic carboxylic acid, a carboxylic acid reductase (CAR), and a ω-transaminase (TA) in the single reaction mixture, reducing the biomass-derived or plastic-derived aryl or heterocyclic carboxylic acid to an aryl or heterocyclic aldehyde, and transferring an amine to the aryl or heterocyclic aldehyde. As a result, the amine product is produced in the single reaction mixture.

The amine product may be produced from a carboxylic acid in a single reaction mixture without any ATP or NADPH cofactor regeneration enzyme. Such a single reduction mixture may comprise 2-[4-(2-Hydroxyethyl) piperazin-1-yl]ethane-1-sulfonic acid (HEPES) (or a similar buffer) at about pH 7.5, dimethylsulfoxide (DMSO), ATP disodium salt, NADPH tetrasodium salt, magnesium chloride (MgCl₂), pyridoxal 5′-phosphate (PLP), and isopropylamine (iPr-NH-2).

The amine product may also be produced from a carboxylic acid in a single reaction mixture with ATP and NADPH cofactor regeneration enzymes. Such a single reduction mixture may comprise polyphosphate kinase type 2-III, glucose dehydrogenase, inorganic pyrophosphatase, HEPES (or a similar buffer) at about pH 7.5, 5% DMSO, adenosine 5′-monophosphate (AMP) monosodium salt, a polyphosphate (polyP) source such as sodium hexametaphosphate (SHMP), a NADP disodium salt, glucose, MgCl₂, pyridoxal 5′-phosphate (PLP), and isopropylamine (iPr-NH-2).

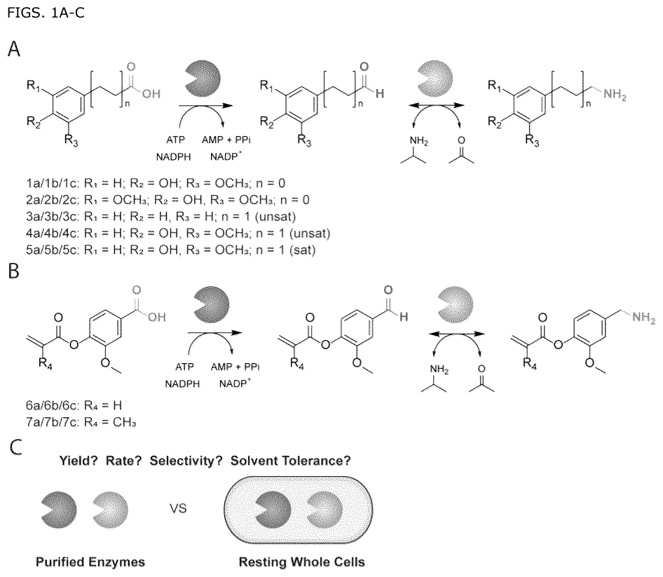

In one embodiment as shown in FIG. 1A, the biomass-derived aryl carboxylic acid is a guaiacol and syringol 4-substituted carboxylic acid of Formula I

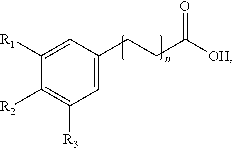

The guaiacol and syringol 4-substituted carboxylic acid is reduced to the aryl aldehyde of Formula II

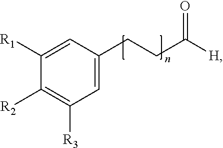

in the presence of ATP, NADPH and Mg²⁺ ions. The CAR catalyzes the reduction. An amine is transferred to the aryl aldehyde of Formula II to produce the amine product of Formula III

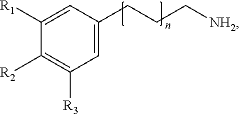

The amine transfer is catalyzed by the ω-TA.

The guaiacol and syringol 4-substituted carboxylic acid of Formula I may be compound 1a, the aryl aldehyde of Formula II may be compound 1b, and the amine product of Formula III may be compound 1c. In 1a, 1b and 1c, R₁ is H, R₂ is OH, R₃ is OCH₃, and n is 0.

The guaiacol and syringol 4-substituted carboxylic acid of Formula I may be compound 1a, the aryl aldehyde of Formula II may be compound 1b, and the amine product of Formula III may be compound 1c. In a1, b1 and c1, R₁ is H, R₂ is OH, R₃ is OCH₃, and n is 0.

The guaiacol and syringol 4-substituted carboxylic acid of Formula I may be compound 2a, the aryl aldehyde of Formula II may be compound 2b, and the amine product of Formula III may be compound 2c. In a2, b2 and c2, R₁ is OCH₃, R₂ is OH, R₃ is OCH₃, and n is 0.

The guaiacol and syringol 4-substituted carboxylic acid of Formula I may be compound 3a, the aryl aldehyde of Formula II may be compound 3b, and the amine product of Formula III may be compound 3c. In a3, b3 and c3, R₁ is H, R₂ is H, R₃ is H, and n is 1 (unsaturated).

The guaiacol and syringol 4-substituted carboxylic acid of Formula I may be compound 4a, the aryl aldehyde of Formula II may be compound 4b, and the amine product of Formula III may be compound 4c. In a4, b4 and c4, R₁ is H, R₂ is OH, R₃ is OCH₃, and n is 1 (unsaturated).

The guaiacol and syringol 4-substituted carboxylic acid of Formula I may be compound 5a, the aryl aldehyde of Formula II may be compound 5b, and the amine product of Formula III may be compound 5c. In a5, b5 and c5, R₁ is H, R₂ is OH, R₃ is OCH₃, and n is 1 (saturated).

In another embodiment as shown in FIG. 1B, the biomass-derived aryl carboxylic acid is of Formula IV

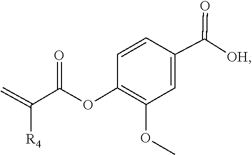

The biomass-derived aryl carboxylic acid of Formula IV is reduced to the aryl aldehyde of Formula V

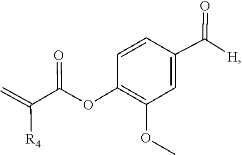

in the presence of ATP and NADPH. The reduction is catalyzed by the CAR. An amine is transferred to the aryl aldehyde of Formula V to produce the amine product of Formula VI

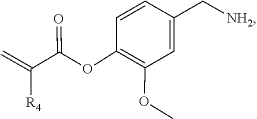

The amine transfer is catalyzed by the ω-TA.

The biomass-derived aryl carboxylic acid of Formula IV may be compound 6a, the aryl aldehyde of Formula V may be compound 6b, and the amine product of Formula VI may be compound 6c. In 6a, 6b and 6c, R₄ may be H.

The biomass-derived aryl carboxylic acid of Formula IV may be compound 7a, the aryl aldehyde of Formula V may be compound 7b, and the amine product of Formula VI may be compound 7c. In 7a, 7b and 7c, R₄ may be CH₃.

The biomass-derived aryl carboxylic acid may be a furan carboxylic acid. The furan carboxylic acid may be 2,5-dicarboxylic acid or 2-furoic acid.

The plastic-derived aryl carboxylic acid may be a polyethylene terephthalate (PET)-derived aryl carboxylic acid. The PET-derived aryl carboxylic acid may be selected from the group consisting of terephthalic acid (TPA), 4-formylbenzoic acid (4FBA), mono-(2-hydroxyethyl)-terephthalic acid (MHET) and monomethyl terephthalate (mmTPA).

According to the single-reaction preparation method, the PET-derived aryl carboxylic acid may be terephthalic acid (TPA) and the amine product may be para-xylylenediamine (pXYL). The single-reaction preparation method may further comprise producing an intermediate selected from the group consisting of 4FBA, TPAL, pAMBA and pAMB. The single-reaction preparation method may further comprise converting the TPA to 4FBA, converting the 4FBA to TPAL, converting the TPAL to pAMB, and converting the pAMB to the pXYL. The single-reaction preparation method may further comprise converting the TPA to 4FBA, converting the 4FBA to pAMBA, converting the pAMBA to pAMB, and converting the pAMB to the pXYL. The pXYL may have a molar yield of about 1-90%, 10-80%, 20-80%, 20-70%, 20-60%, 20-50%, 20-40%, 20-30%, 30-80%, 30-70%, 30-60%, 30-50%, 30-40%, 40-80%, 40-70%, 40-60%, 50-80%, 50-60%, 60-90%, 70-90% or 80-90%, or at least 20%, 30%, 40%, 50%, 60%, 70%, 80%, 90% or 100% of the TPA as supplied.

According to the single-reaction preparation method, the PET-derived aryl carboxylic acid may be mono-(2-hydroxyethyl)-terephthalic acid (MHET) and the amine product may be para-(aminomethyl)benzoic acid (pAMBA). The single-reaction preparation method may further comprise producing mono-(2-hydroxyethyl)-para-(aminomethyl)benzoic acid (MHE-pAMBA) as an intermediate. The single-reaction preparation method may further comprise converting the MHET to mono-(2-hydroxyethyl)-para-(aminomethyl)benzoic acid (MHE-pAMBA), and converting the MHE-pAMBA to the pAMBA.

The ω-TA may consist of an amino acid sequence having at least 80% identity to the amino acid sequence of SEQ ID NO: 18 (Table 1). The ω-TA may be cvTA (SEQ ID NO: 18).

The CAR may be selected from the group consisting of an amino acid sequence having at least 80% identity to an amino acid sequence selected from the group consisting of SEQ ID NOs: 1-17 (Table 1). The CAR may be selected from the group consisting of an amino acid sequence having at least 80% identity to an amino acid sequence selected from the group consisting of SEQ ID NOs: 1-16. The CAR may be a CAR from Segniliparus rotundus (srCAR) (SEQ ID NO: 15), a CAR from Trichoderma reesei (trCAR) (SEQ ID NO: 13), a CAR from Mycolicibacterium smegmatis (msCAR) (SEQ ID NO: 10), a CAR from Mycobacterium avium (mavCAR) (SEQ ID NO: 12), a CAR from Neurospora crassa (ncCAR) (SEQ ID NO: 14), a CAR from Aspergillus fumigatus (afCAR) (SEQ ID NO: 2) or a CAR from Nocardia iowensis (niCAR) (SEQ ID NO: 16).

Where the PET-derived aryl carboxylic acid is TPA and the amine product is para-xylylenediamine (pXYL), the CAR may be srCAR, trCAR, msCAR, mavCAR, ncCAR or afCAR, and the ω-TA may be cvTA.

Where the PET-derived aryl carboxylic acid is mmTPA, the CAR may be srCAR or niCAR, and the ω-TA may be cvTA.

Where the PET-derived aryl carboxylic acid is MHET and the amine product is para-(aminomethyl)benzoic acid (pAMBA), the CAR may be niCAR, srCAR or trCAR, and the ω-TA may be cvTA.

According to the single-reaction preparation method, the CAR may be expressed by recombinant cells. The recombinant cells may be recombinant cells. The CAR may be purified from the recombinant cells. The single reaction mixture may comprise the purified CAR. The single reaction mixture may comprise the recombinant cells expressing the CAR.

According to the single-reaction preparation method, the ω-TA may be expressed by recombinant cells. The recombinant cells may be recombinant cells. The ω-TA may be purified from the recombinant cells. The single reaction mixture may comprise the purified ω-TA. The single reaction mixture may comprise the recombinant cells expressing ω-TA.

According to the single-reaction preparation method, the CAR and the ω-TA may be expressed by recombinant cells. The recombinant cells may be recombinant cells. The CAR and the ω-TA may be purified from the recombinant cells. The single reaction mixture may comprise the purified CAR and ω-TA. The single reaction mixture may comprise the recombinant cells expressing the CAR and the ω-TA.

The present invention also provides a method for reducing a substrate. This reduction method comprises incubating the substrate and a carboxylic acid reductase (CAR) in a reduction mixture to produce a reduction product. The substrate is selected from the group consisting of terephthalic acid (TPA), 4-formylbenzoic acid (4FBA), mono-(2-hydroxyethyl)-terephthalic acid (MHET), monomethyl terephthalate (mmTPA), and para-(aminomethyl)benzoic acid (pAMBA). The CAR consists of an amino acid sequence having at least 80% identity to an amino acid sequence selected from the group consisting of SEQ ID NOs: 1-17.

The reduction mixture may be prepared without a cofactor regeneration enzyme. In this case, the reduction mixture may comprise HEPES (or a similar buffer) at about pH 7.5, DMSO, ATP, NADPH, and 10-20 mM MgCl₂.

The reduction mixture may also be prepared with cofactor regeneration enzymes. In this case, the reduction mixture may comprise polyphosphate kinase 2-III, which is an ATP regeneration enzyme, glucose dehydrogenase for NADPH regeneration, inorganic Pyrophosphatase for pyrophosphate removal, HEPES (or a similar buffer) at pH about 7.5, 5% DMSO, adenosine 5′-monophosphate, a polyphosphate (polyP) source such as sodium hexametaphosphate, NADP, glucose, and 10-20 mM MgCl2.

According to the reduction method, the CAR may consist of an amino acid sequence selected from the group consisting of SEQ ID NOs: 1-17. The CAR may consist of an amino acid sequence having at least 80% identity to an amino acid sequence selected from the group consisting of SEQ ID NOs: 1-16. The CAR may consist of an amino acid sequence selected from the group consisting of SEQ ID NOS: 1-16. The CAR may be a CAR from Segniliparus rotundus (srCAR) (SEQ ID NO: 15), a CAR from Trichoderma reesei (trCAR) (SEQ ID NO: 13), a CAR from Mycolicibacterium smegmatis (msCAR) (SEQ ID NO: 10), a CAR from Mycobacterium avium (mavCAR) (SEQ ID NO: 12), a CAR from Neurospora crassa (ncCAR) (SEQ ID NO: 14), a CAR from Aspergillus fumigatus (afCAR) (SEQ ID NO: 2) or a CAR from Nocardia iowensis (niCAR) (SEQ ID NO: 16). The CAR may be srCAR (SEQ ID NO: 15). The CAR may be trCAR.

According to the reduction method, the substrate may be TPA and the reduction product may be 4FBA or terephthalaldehyde (TPAL). The CAR may be srCAR, trCAR), msCAR, mavCAR, ncCAR or afCAR.

According to the reduction method, the substrate may be mmTPA and the reduction product may be para-(aminomethyl)benzoic acid (pAMBA). The CAR may be srCAR or niCAR.

According to the reduction method, the substrate may be MHET and the reduction product may be 2-hydroxyethyl 4-formylbenzoate (MHE-4FBA). The CAR may be niCAR, srCAR or trCAR.

According to the reduction method, the substrate may be 4FBA and the reduction product may be terephthalaldehyde (TPAL).

According to the reduction method, the substrate may be pAMBA and the reduction product may be para-aminomethylbenzaldehyde (pAMB).

According to the reduction method, the CAR may be expressed by recombinant cells. The recombinant cells may be recombinant cells. The CAR may be purified from the recombinant cells. The reduction mixture may comprise the purified CAR. The reduction mixture may comprise the recombinant cells, for example, recombinant cells, expressing the CAR.

The present invention further provides a method for transferring an amine to an aldehyde. This amine transferring method comprises incubating the aldehyde and an amine transferring enzyme in an amine transferring mixture to produce an amine product. The amine transferring enzyme may be selected from the group consisting of a ω-transaminase (TA), an amine transaminase and a reductive aminase.

Where the amine transferring enzyme is a ω-TA, the amine transferring mixture may comprise HEPES (or a similar buffer) at about pH 7.5, 5% DMSO, pyridoxal 5′-phosphate (PLP), and an amine donor. The amine donor may be isopropylamine (iPr-NH-2). Where the amine transferring enzyme is cvTA, alanine or other amine donors may also be used.

The ω-TA may consist of an amino acid sequence having at least 80% identity to the amino acid sequence of SEQ ID NO: 18 (Table 1). The ω-TA may be cvTA (SEQ ID NO: 18).

According to the amine transferring method, the aldehyde may be 4-formylbenzoic acid (4FBA) and the amine product may be para-(aminomethyl)benzoic acid (pAMBA). The aldehyde may be terephthalaldehyde (TPAL) and the amine product may be para-aminomethylbenzaldehyde (pAMB). The aldehyde may be para-aminomethylbenzaldehyde (pAMB) and the amine product may be para-xylylenediamine (pXYL). The aldehyde may be terephthalaldehyde (TPAL) and the amine product may be para-xylylenediamine (pXYL). The aldehyde may be methyl 4-formylbenzoate (mm4FBA) and the amine product may be methyl 4-(aminomethyl)benzoate (mm-pAMBA). The aldehyde may be 2-hydroxyethyl 4-formylbenzoate (MHE-4FBA) and the amine product may be mono-(2-hydroxyethyl)-para-(aminomethyl)benzoic acid (MHE-pAMBA). The aldehyde may be a furan aldehyde. The furan aldehyde may be furfural or diformylfuran.

According to the amine transferring method, the ω-TA may be expressed by recombinant cells. The recombinant cells may be recombinant cells. The ω-TA may be purified from the recombinant cells. The amine transferring mixture may comprise the purified ω-TA. The amine transferring mixture may comprise the recombinant cells, for example, recombinant cells, expressing ω-TA.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Conversion of Lignin-Derivable Small Molecules to Amine Products

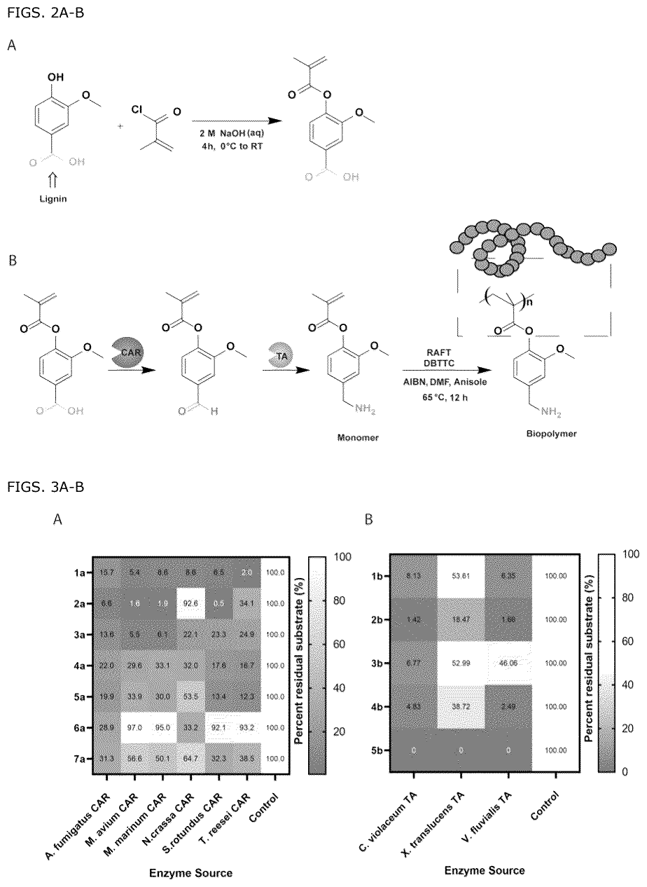

A route to repurpose lignin-derivable small molecules to amine-containing monomers and subsequently polymers was designed. The lignin-derived small molecules are used to synthesize methacrylated vanillate chemically (FIG. 2A), which may be valorized as sustainable alternatives to petroleum-based feedstocks by a CAR and a ω-TA for producing thermostable polymers (FIG. 2B).

The specificity of CAR and ω-TA on lignin-derivable small molecules were explored. The substrate specificity was analyzed in vitro with a naturally occurring CAR to produce lignin-derivable aldehydes or a ω-TA enzyme for converting these aldehydes to amines (FIGS. 3A-B). For all desired chemistries, naturally occurring CAR variants were identified as being capable of catalyzing the selective reduction of a carboxylic acid to its corresponding aldehyde, for example, in FIGS. 1A-B. One variant of interest, the CAR from A. fumigatus (afCAR) (SEQ ID NO: 2), exhibited activity on all the substrates of interest, including the acrylated and methacrylated derivatives which only a limited number of CARs were able to accept. Another CAR from S. rotundus exhibited activity on all but 6a (methacrylated vanillate). Because it exhibited greater activity towards the model compound 1a, this enzyme was used for cascade optimization experiments. Among the aldehydes, 1b-5b were all efficiently converted to their corresponding amine by the ω-transaminase from C. violaceum. As 6b and 7b could not be chemically synthesized in pure form, their relevance was tested for the amine transfer step in a coupled assay later in this work. For the conversion of aryl acid to aldehyde, 0.2 mg/ml of respective CAR was mixed in a reaction mixture containing 0.1 M HEPES pH 7.5, 10 mM MgCl₂, 1×NADPH, 1.25×ATP, and the substrate depletion was measured using RP-HPLC at a desired endpoint 24 h (except 1 h for 6a and 7a due to hydrolysis). For the conversion of aryl aldehydes to amines, 0.5 mg/ml of a respective ω-TA was used in a reaction mixture containing 0.1 M HEPES PH 7.5, 2 mM PLP, and 4×iPr-NH₂ as the amine donor for 24 h at 30° C. The substrate depletion was monitored using RP-HPLC. Thus, efficient conversion of carboxylic acid and aldehyde functional group to amines has been demonstrated.

### Example 2. One-Pot Bioconversion of Lignin-Derivable Carboxylic Acids to Heterocyclic Primary Amines

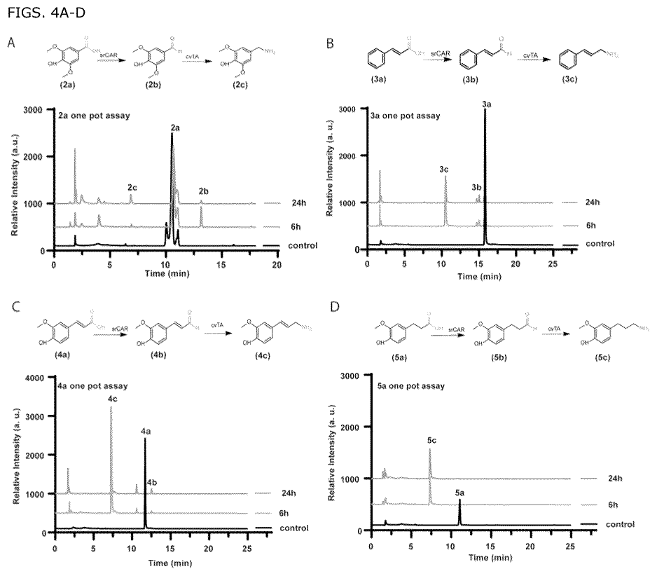

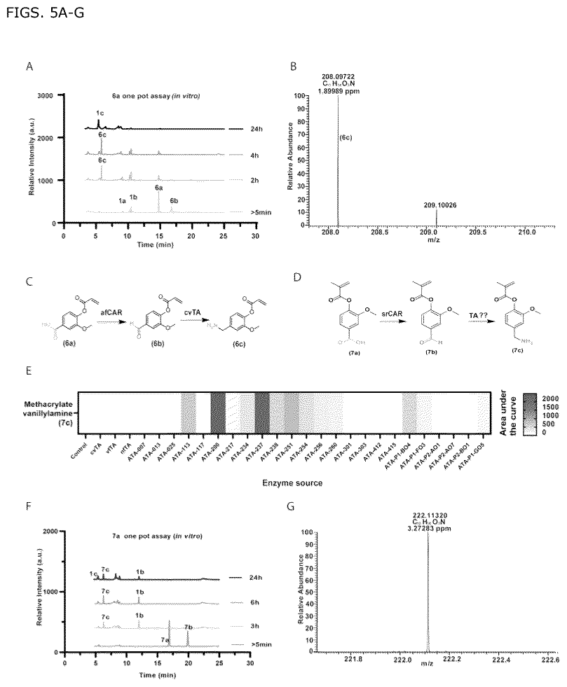

Whole-cell, one-pot bioconversions of lignin-derivable carboxylic acids to heterocyclic primary amines were carried out. HPLC chromatogram traces show a control under reaction conditions for 24 h without whole cell addition (black) and one-pot assay samples after 6 h and 24 h To synthesize these versatile amines, resting whole cell biocatalysts of WC5 (100 mg/mL wcw) containing overexpressed srCAR, bsSfp, cvTA, and AlaDH were incubated in 0.1 M HEPES at 30° C., supplemented with 10 mM of glucose, 8×iPr-NH₂, 10 mM MgCl₂, 10× NH₄Cl, 10% DMSO, and 2 mM PLP at 5 mM of 2a, 3a, 4a, or 5a substrate loading. In all the cases, control involved a 24 h reaction mixture without whole cells. The HPLC traces showing the control, 6 h, and 24 h results of the 2a one-pot assay (FIG. 4A) indicate that 2a is not an effective substrate. The HPLC traces depicting the control, 6 h, and 24 h results of the 3a one-pot assay (FIG. 4B) demonstrate the high efficiency of the whole cell biocatalyst system in converting 3a to 3c, resulting in a high yield of the product within 6 h. The HPLC traces of control, 6 h, and 24 h results (FIG. 4C) demonstrate effective conversion of 4a to 4c. The HPLC traces of a 5a one-pot assay (FIG. 4D) show highly efficient conversion of 5a to 5c within 6 hours, with complete consumption of the substrate and no detectable byproducts; the only peak observed corresponds to the desired product, 5c.

### Example 3. One-Pot Bioconversion of Acrylated and Methacrylated Compounds to Amines

One-pot enzyme cascade strategies for the bioconversion of acrylated and methacrylated compounds to amines were designed. HPLC chromatogram traces show the progress of a one-pot enzyme cascade reaction for the bioconversion of 6a to 6c (FIG. 5A). The reaction was performed in 0.1 M HEPES (pH 7.5) with purified enzymes afCAR, ecPPase, and cvTA, 8×iPr-NH₂, 1×NADPH, 1.25×ATP, 10 mM MgCl₂, 2 mM PLP and 5 mM 6a substrate loading at 30° C. The chromatogram traces demonstrate fast reaction kinetics and gradual hydrolysis over time, up to 24 hours. 6c synthesis at 4 hours was confirmed by mass spectra result from the one-pot assay conducted in panel A (FIG. 5B). FIG. 5C illustrates a reaction schematic for the one-pot reduction and amination of 6a to 6c. FIG. 5D illustrates a reaction schematic for the one-pot reduction and amination of 7a to 7c. FIG. 5E shows a heatmap of a one-pot enzyme cascade reaction for the bioconversion of 7a to 7c. The reaction was carried out in 0.1 M HEPES (pH 7.5) containing 8×iPr-NH₂, 1×NADPH, 1.25×ATP, and 10 mM MgCl₂ for in-house ω-TAS (cvTA, vfTA, and xtTA). The Codex is recommended protocol was used for all ATAs along with purified enzymes srCAR, ecPPase, and 5 mM 7a substrate loading at 30° C. The HPLC chromatogram traces show the progress of a one-pot enzyme cascade reaction for the bioconversion of 7a to 7c (FIG. 5F). The reaction was carried out in 0.1 M HEPES (pH 7.5) containing 0.1M iPr-NH₂, 1×NADPH, 1.25×ATP, 10 mM MgCl₂ using srCAR, ecPPase, and ATA-200 at 30° C. 7c synthesis at 6 hours was confirmed by mass spectra result (FIG. 5G) from the one-pot assay conducted in panel F.

### Example 4. Conversion of Furans Derived from Deconstruction Products of PEF or Lignocellulosic Biomass

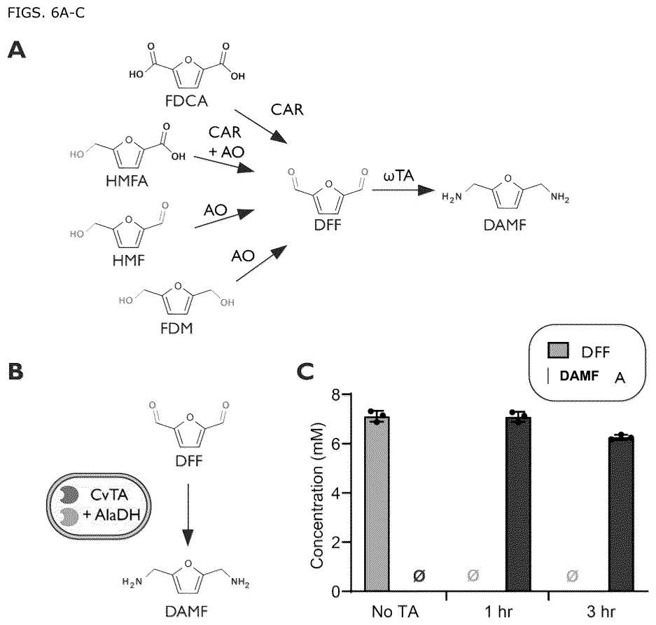

Furans were derived from deconstruction products of PEF or lignocellulosic biomass. FIG. 6A shows a reaction schematic for the conversion of furan starting compounds to FDMA by an enzyme cascade consisting of CARs or aldehyde oxidases (AOs) and ω-TAs. ω-TA from Chromobacterium violaceum (CvTA) with an alanine dehydrogenase from Bacillus subtilis (BsAlaDH) were used for the conversion of DFF to 2,5-furan-dimethylamine (FDMA, otherwise known as DAMF or 2,5-diaminomethylfuran) in resting whole cells (FIG. 6B). To obtain synthesis of DAMF, resting whole cell biocatalysis of ROAR (50 mg/ml wet cell weight) containing overexpressed CvTA and BsAlaDH were incubated at 30° C. with 40 mM isopropylamine used as an amine donor and 5 mM DFF (FIG. 6C).

### Example 5. Strategies for Valorizing Deconstruction Products of PET

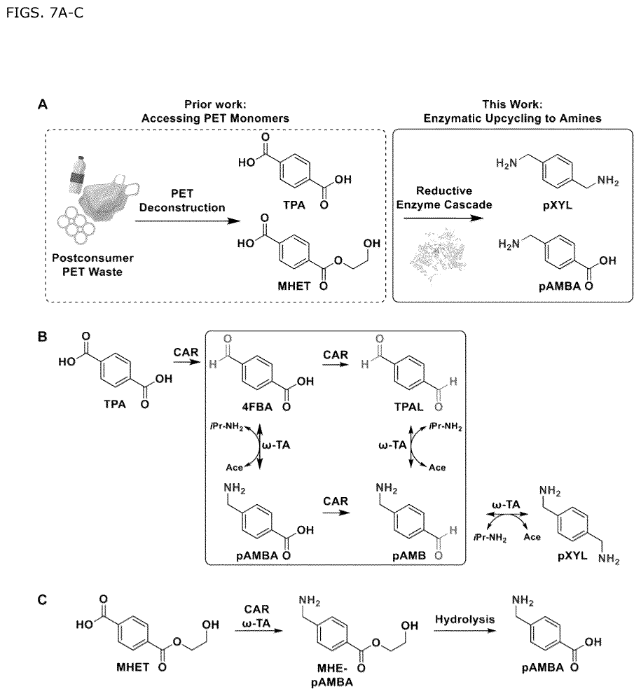

Using this enzyme cascade is envisioned to upcycle PET waste by creating value-added amines for downstream application in therapeutics and polymer synthesis (FIG. 7A). The reaction schematic for the one-pot reduction and amination of TPA to pXYL by an enzyme cascade consisting of carboxylic acid reductases (CAR) and ω-transaminases (ω-TA). The reaction cascade goes through four potential intermediate compounds, 4FBA, TPAL, pAMBA, and pAMB, due to successive reduction and amination reactions (FIG. 7B). The substrate specificities of CAR and ω-TA could allow functionalization of MHET to pAMBA, a small-molecule therapeutic, by a simple one-pot enzymatic cascade (FIG. 7C). iPr-NH₂, isopropylamine; CAR, carboxylic acid reductase; ω-TA, ω-transaminase; Ace, acetone; TPA, terephthalic acid; 4FBA, 4-formylbenzoic acid; TPAL, terephthalaldehyde; pAMBA, para-(aminomethyl)benzoic acid; pAMB, para-aminomethylbenzaldehyde; pXYL, para-xylylenediamine.

### Example 6. Testing cvTA on PET-Derived Aldehydes to Produce Amines

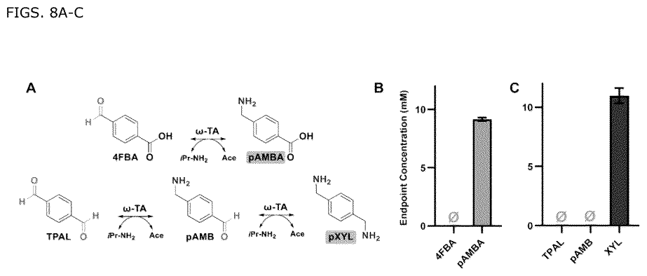

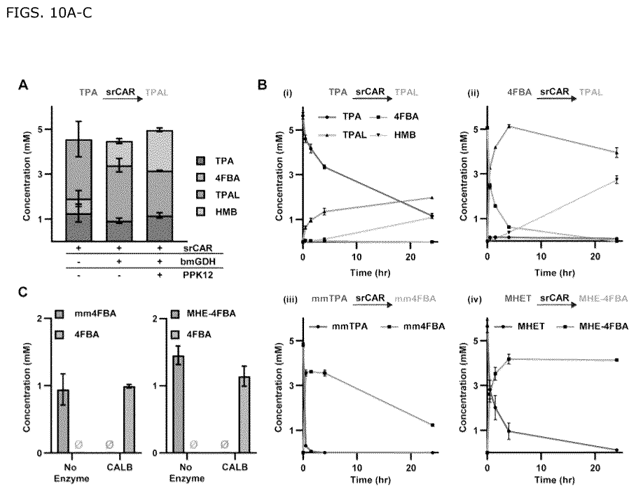

cvTA reactions for the conversion of 4FBA and TPAL to their corresponding amine (FIG. 8A) Endpoint bioconversion of 10 mM 4FBA to pAMBA (FIG. 8B) and 10 mM TPAL to pXYL (FIG. 8C) by 1.5 μM cvTA in 100 mM HEPES pH 7.5, 400 μM PLP with 40 mM iPr-NH₂ as the amine donor. This reaction proceeds to completion after 24 h at 30° C., with no detection of aldehyde reactants or intermediates after 24 h. Sample size is N=3 and all data are shown as the mean±standard deviation.

### Example 7. Evaluating CAR Specificity on Products of PET Deconstruction

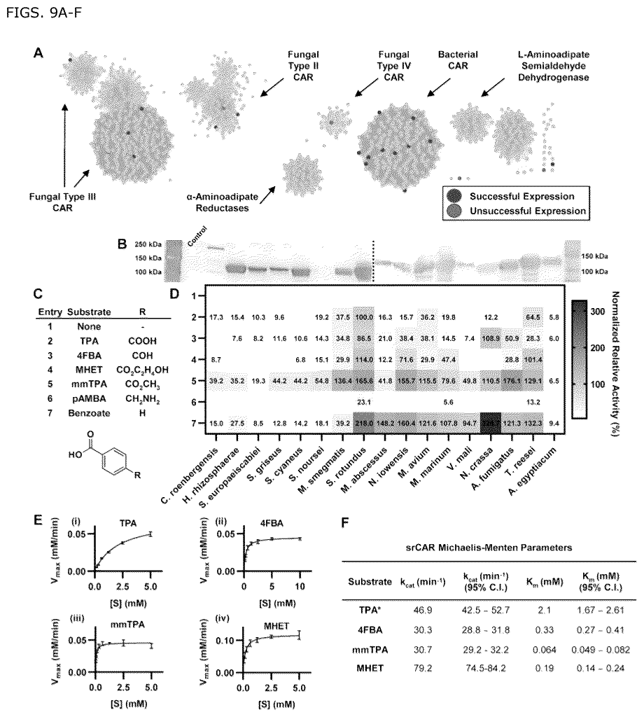

Using a sequence similarity network, CARs were sampled across distinct protein clusters to probe for activity on TPA. Of 22 cloned CARs, 17 expressed, with 9 of those CARs being unreported in literature (FIG. 9A). Anti-His₆ Western blot of cell lysate from E. coli BL21 (DE3) harboring different CARs is shown by phylogeny (FIG. 9B). CARS were expressed under optimized conditions. The Western blot is comprised of two separate blots, which are delineated by the dashed lines and contain their own protein ladders. The first lane of the Western blot is a control lane consisting of lysate from E. coli BL21 (DE3) with no CAR plasmid. A band corresponding to the putative CAR from Streptomyces noursei was not detected during Western blot analysis but was confirmed in a separate experiment. The substrate specificity of CAR was evaluated on TPA, 4FBA, MHET and mmTPA, which are carboxylates related to PET depolymerization (FIG. 9C). In addition, pAMBA was tested, as it is a potential reaction intermediate in our proposed multienzyme cascade for the formation of amines. Under mild aqueous conditions, the conversion of CAR was tracked on TPA, 4FBA, MHET, mmTPA, pAMBA and the model aromatic acid, benzoic acid (FIG. 9D). Here, the oxidation of NADPH is measured as a proxy to aldehyde formation, as depletion of NADPH is linked to the consumption of substrate. The heatmap was generated by calculating the triplicate average of initial rates of each CAR-substrate pair. The heatmap values represent the average initial rate, normalized to the average activity of srCAR on TPA of 4.80 mM NADPHₒₓ·h⁻¹·μM_{CAR}⁻¹. CARs-substrate pairs with <5% normalized relative activity are not shown. A Michaelis-Menten study was performed on the highest performing CAR from Segniliparus rotundus to evaluate k_{cat} and Kₘ on TPA (i), 4FBA (ii), MHET (iii) and mmTPA (iv) (FIG. 9E). Michaelis-Menten parameters and 95% confidence intervals were fitted using non-linear least squares regression on Graphpad Prism v9.3.1 (FIG. 9F). Sample size is N=3 for all numerical data. *srCAR kinetic parameters on TPA are reported as apparent values due to difficulty decoupling monofunctional and bifunctional reduction.

### Example 8. Reduction of PET-Derived Deconstruction Products Catalyzed by srCAR

Endpoint in vitro assays showing the conversion of 5 mM TPA to TPAL after 24 h, with and without co-factor regenerating enzymes. Our multienzyme cascade shows the overreduction of TPA to an alcohol, 4-(hydroxymethyl)benzaldehyde (HMB) (FIG. 10A). Time course analysis of the enzymatic reduction of (i) 5 mM TPA, (ii) 5 mM 4FBA, (iii) 5 mM mmTPA and (iv) 5 mM MHET by CAR (FIG. 10B) All experiments included a 3-enzyme NADPH and ATP regeneration system. Reduction of TPA and 4FBA show over-reduction of aldehydes to alcohols. While mmTPA shows a decline in concentration over time to the purported alcohol product, this was not independently verified. Surprisingly, MHET shows no over-reduction to the alcohol. At our chosen endpoint of 24 h, lipase from Candida antarctica (CALB) completely de-esterified 1 mM of both MHE-4FBA and mm4FBA at 30° C., providing an alternate route to forming aldehydes from PET-derived esters (FIG. 10C). Sample size is N=3 and all data are shown as the mean±standard deviation. Error bars for certain points of the bioconversion of mmTPA to mm4FBA are smaller than the height of the symbol.

### Example 9. One-Pot Bioconversion of PET-Derived Carboxylates to Amines

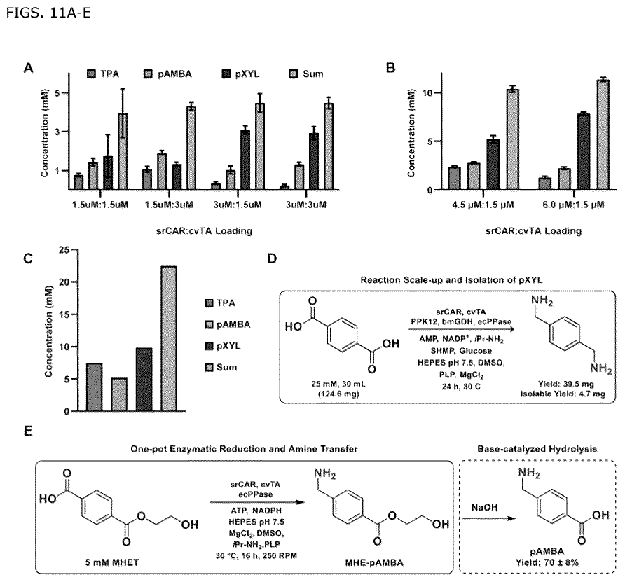

One-pot in vitro bioconversion of TPA to pXYL by a 5-enzyme cascade consisting of srCAR, cvTA and a 3-enzyme co-factor regeneration cascade yields high reaction selectivity for amines and an appreciable molar yield of 62% of the desired diamine pXYL at 3 μM srCAR loading. By tuning the ratio of CAR to TA, a corresponding increase was observed in yield and selectivity towards the diamine when CAR concentration is increased (FIG. 11A). At 10 mM loading of TPA as substrate, 69±1% molar yield of TPA to pXYL in one-pot was demonstrated at a srCAR:cvTA ratio of 6.0 μM: 1.5 μM (FIG. 11B). Operating at the same 6.0 μM: 1.5 μM srCAR:cvTA ratio used in the 10 mM experiment, the reaction was scaled up to 30 mL volume at 25 mM substrate loading, which corresponds to 100 mg theoretical yield of pXYL. 6.0 μM srCAR, 1.5 μM cvTA, 1.5 μM bmGDH (tetramer basis), 0.84 mg/mL PPK12, and 1.5 μM ecPPase (hexamer basis) were added in pH adjusted buffer that contained 100 mM HEPES pH 7.5, 1 mM AMP, 1 mM NADP⁺, 100 mM iPr-NH₂, 15 mM SHMP, 50 mM glucose, 5% v/v DMSO, 2 mM PLP, and 30 mM MgCl₂, and 25 mM TPA. The reaction proceeded for 24 h at 30° C. and was mixed by inversion with a rotating rotisserie. After 24 h, endpoint HPLC showed 9.8 mM, or 39.5 mg, pXYL in solution. As expected, pAMBA was also present at 5.2 mM, and unreacted TPA comprised 7.5 mM of the reaction. This experiment was conducted in N=1 (FIG. 11C). After performing a two-phase organic extraction, 4.7 mg of pXYL was isolated, even though the overall reaction yielded 39.5 mg, as quantified by HPLC (FIG. 11D). The substrate specificity of srCAR and cvTA allows us to produce the pharmaceutically relevant monoamine, pAMBA, solely by switching the starting substrate from TPA to MHET, and performing a simple hydrolysis step to liberate pAMBA, yielding 70±8% of the desired product. For the bioconversion of MHET to pAMBA, 6.0 M srCAR, 1.5 μM cvTA (dimer basis), 0.75 μM ecPPase (hexamer basis), 6 mM NADPH, 6 mM ATP, 20 mM MgCl₂, 100 mM HEPES pH 7.5, 5% (v/v) DMSO, 20 mM 10 iPr-NH₂, 400 μM PLP, and 5 mM of MHET were added to a total volume of 100 μL. Following a 16 h incubation at 30° C., the reaction was basified by NaOH to a final concentration of 0.5 M and analyzed using HPLC-UV (FIG. 11E). Sample size for panels A, B, and E is N=3. Error bars are shown as the mean±standard deviation.

TABLE 1
Enzymes

| Organism/ | Gene | Plasmid | | SEQ |
|---|---|---|---|---|
| Enzyme | Accession | Accession | | ID |
| (Abbreviation) | Number | Number | Protein AA Sequence | NO: |
| Valsa mali/ | KUI69596.1 | OP031622 | MGSSHHHHHHMGSVANSENVPYGKR | 1 |
| CAR | | | LLPHIVDEVASKDPNRECIQIPWSSEP | |
| (vmCAR) | | | SDGWRTITWKDMANGINRCAHRIVEL | |
| | | | FGTPQEGSFPTIAYIGPNDARYVLMMI | |
| | | | AASKAGYKALFISPRNSKEGQINLFKT | |
| | | | TDCNILAFASSHKDTIKPWIAEHSMQT | |
| | | | FEVSPMDSWFPDTEVPHFTYTKTFEDA | |
| | | | EWDPLLVLHTSGSTGLPKPIVVKQGM | |
| | | | WAGMDAYHNLPKFEGKNVAMREWAE | |
| | | | RAKRHFIPMPLFHIAGLLSVVNLSLYFD | |
| | | | TPVALGIPDRPLSADNVIECLENLDVES | |
| | | | AFLPPAVIEDMSQTEEGTKVLTSLNVV | |
| | | | AFGGGNLAREAGNRLVKQGAVFMNLI | |
| | | | GSTEFAPFPTYARNDPEAWQYFVYNPN | |
| | | | VMGCEFRKQGDEDVYEMVVVRQNKH | |
| | | | PGLQGIFYTFPELNEWSSRDLYRPHPT | |
| | | | KPYHWIYHGRADNIIVFSNGEKLNPVT | |
| | | | IEEIVSDHPSLKGALVVGSEKFQAGLII | |
| | | | EPYETPKNAAEEQALLDSVWPLVERAN | |
| | | | EETVAHGRISRDMIRLSNPQKPFLRAA | |
| | | | KGTVQRAGTIKLYADEIEKLYEKESSD | |
| | | | QDQDAPKLDISSEDALTASIVDALRKD | |
| | | | VGADKLDADVDFFAAGIDSLGVMRAA | |
| | | | KLLRAGLKEAGHEVDAKSFATRVIYQN | |
| | | | STPRRLASHILNAFINGQGESLSEDEQ | |
| | | | QIQAMEVIWNKYTSNLTKAVPNRPDP | |
| | | | NTKDQTVILTGSTGMLGSYLLDFMGR | |
| | | | NPRVSKIICFNRAADGGRAQGLKACVE | |
| | | | RGLDATALKTKAEFYQVDLSQPKLGLS | |
| | | | DDVYARLQAETDRVIHNAWPVNFNIPI | |
| | | | ESFDPSLAGVRHIADLAATAAKRVAVT | |
| | | | FIASIAVADRWQEDGKVPETRLEELAL | |
| | | | ANGGYGRSKMVGSLILEDAAAKGAGD | |
| | | | FPYAIVRVGQIAGPEGEAGEWNKHEW | |
| | | | LPSIIASSLYLHALPKNLGTMDRVDWT | |
| | | | PVERVAKLVLEASGTARVVQSADEING | |
| | | | YYHGVNPNYTQWYDLAVAVQEFYGKD | |
| | | | KIPEFISFSEWVDRLEKSQADGPESVA | |
| | | | ANPGVKLIDTYRAMASADKAVVYDMQ | |
| | | | KTLERCPVVKETKAITPEMMKHWCAQ | |
| | | | WRH* | |
| Aspergillus | XP_748589.2 | OP031615 | MGSSHHHHHHMEAVVDTTSETAKPP | 2 |
| fumigatus/ | | | QRLLATVVDSLALECPTRRFCLIPNGK | |
| CAR | | | DVHQGFREVTFRDLCCAVNRMSWWM | |
| (afCAR) | | | EKHLASSVKGATIAYLGSNDIRYIILML | |
| | | | ASHKTGCTIFFPSTRLSNEAYDSVFGA | |
| | | | TQTKMLLFSPEKHPLVSGLTGPSKAIS | |
| | | | SLEVPSVPEMLNDNPDVKNYPFTSTFE | |
| | | | EFEDKTAFIIHSSGTTGMPKPVSLTHG | |
| | | | YLGTVDYSAFMPRPAGRSPSFFQDLLS | |
| | | | ADPHPRDPVLSVTPYFHIMGLVSFFVSI | |
| | | | FHNIPFVTISDQPLSVSLLVDIIRATHPT | |
| | | | ATILPPSILEDMSLSQEALECLGTLKFV | |
| | | | CYGGAPLAKQVGDKVSQYTQLRNAIG | |
| | | | STEIGIIGSLVPEGKENWGYFEWNPAY | |
| | | | GIDMQPVADDVYELVIPRLEDSRRMH | |
| | | | GIFHTFPSFKEYRSKDLYVRHPKIPKLW | |
| | | | QYRGRLDDVIVLSNGEKLNPVTLEKVV | |
| | | | EGHPFVRRALVFGQGRFQTGLLIEPAM | |
| | | | DDRAGKIDERNFVDMIWPLVQTANQN | |
| | | | VPRYGQVLKNMIRLASPAKPFKLTPKG | |
| | | | TTQRHAVNADYAEEIDAMYATHEKQL | |
| | | | GPKLPSTIDSESVHCYVHEVITSLTGRS | |
| | | | DIRPSDDLFGLGLDSLQATQLSNILRS | |
| | | | AVLSYNPALSTENITVQNIYTRPTTDKL | |
| | | | AGLLLGVLQEQKEQAAIAPTESRSERI | |
| | | | AGLVSKYTADLPARYVNSPTQLPRLST | |
| | | | VILTGSTGSLGTYILSGLLNDPHVAKVY | |
| | | | CFNRAADAATRQRQGFAEKGLDASLL | |
| | | | EDPSKVEFLHVSFGDKHFGLDDSMYS | |
| | | | KLLDTVDLIVHNAWKVNFNHPVSSFE | |
| | | | DPHIKGVREFVNFSLEARYNTHLAFVS | |
| | | | SVSTIAGWTPSSDESAVPELPMDTVD | |
| | | | AVLKQGYGESKHVGERICLEASRTSGV | |
| | | | PTSVLRVGQIAGPDSRLGLWNPHEWL | |
| | | | PSVVKTSKSMGKVPDTLGSVLVDWIA | |
| | | | VDTLAKITIEILLSRRSSLSTQRHAVFH | |
| | | | LTNPSQIPWASLIPAIQERYPMSVVSLA | |
| | | | EWVEELEHIRNPSPQDLAKRPALKLLS | |
| | | | FYQALARNAGAPNAEISVENSKKASRT | |
| | | | MASLGPVSLAQMSNWLNQWDF* | |
| Acremonium | BBF25314.1 | OP031613 | MGSSHHHHHHMTVNGHHTNGVNGA | 3 |
| egyptiacum/ | | | NGTNGHANGSNGINDTKAVKEIVPFV | |
| CAR | | | KPQVNFASAQRLEGCIHSLPELVDFNS | |
| (aeCAR) | | | LNNQHHTFCVQAKSSEPFDTITHGEFK | |
| | | | VAVSKCAAWLKENLPIRPSSDDKALTK | |
| | | | MAPVALFMESDIGLVIHEFALMSIGVPP | |
| | | | LVLSPRLSPVAINALLEATGAASFIVSP | |
| | | | RMSEPLKGALAALAAKGVSTHIGNPYK | |
| | | | AYYQPGADPKSVAPFEVPQNPEDVILL | |
| | | | LHSSGTTGLPKPIPTTHRQLLFAVNCH | |
| | | | KFDTEEQAQSLNLSTLPLFHGFGLVAP | |
| | | | GLSMSAGKPTLYPASDGIPNAKSIVDL | |
| | | | INKTNAKSMMTVPFLLDDITNLPNEEGI | |
| | | | KALVHMDFVGTGGAALGAGIGDRLAK | |
| | | | GGVKLLNFYGTTETGPLSLTFAPTDNY | |
| | | | DWKYFRLRTDCEYKIDELEPRDGERRF | |
| | | | RLTVYPYGSEGFEISDQLIRNEQYPETD | |
| | | | FAAVGRDDDVIVLATGEKANPLILETKL | |
| | | | TEAPMVKAAIAFGENQFNLGVIVEPAE | |
| | | | PLTPDTESAFRESIWPIITAACDQMDA | |
| | | | FSRIPSPDAVVLVPAGVVIPRTDKGSIA | |
| | | | RKETYALFDKQIKGVYEQLLKAAADAV | |
| | | | EPLDLDNLEQNLKSLIQEHLHIQAPAS | |
| | | | DWGVEDSLFDIGVDSLQVLQLRRILVT | |
| | | | AASKTEAFKDTDCEKMIPPEFVYMNPS | |
| | | | IREIAAALTKGSDGGDVSLEDAAKEVV | |
| | | | ELAETYSLKGVSAQEKAPSSSEGAFVM | |
| | | | LTGATGSLGSHVAADLARRDNVAKVV | |
| | | | CLVRKDKGTNQPPMPGGNPFDKKILK | |
| | | | ARGIQLTDEQFGKLATLEVDPTADKLG | |
| | | | LIPMAYGMMQAKVTHVIHAAWPMNYL | |
| | | | IRLRNFQYQFKFLRNLLEFASQGPAPTK | |
| | | | KRFVFISSIATVARIGLAQPGSISEAPV | |
| | | | SPSDSACGIGYADGKLVCEKIMEKAA | |
| | | | QDYGGQLDVTSVRCGQMTGSKKTGV | |
| | | | WNSNEQIPMLLKSAQGLGSLPQLSGE | |
| | | | LSWIPVDDAASTVSEIAFSDGSMPIVQ | |
| | | | HLENPIRQSWDAMLQSFGRELGLPAG | |
| | | | KVPFGEWLDQVAAADGDDETFPVKKL | |
| | | | TFFFKSFFQSVACGQVVLDTTVSRGQS | |
| | | | KTLNAMTAVGDETVKAYADYWKSTGY | |
| | | | LSK* | |
| Herbihabitans | WP_130345796.1 | OP031617 | MGSSHHHHHHMQNPELGLAEVVATV | 4 |
| rhizosphaerae/ | | | MDGYADRPAVGDRVTEKVTDPATGRT | |
| CAR | | | TLRLLPQFDTLTYREVWRHAGAIASAW | |
| (hrCAR) | | | QHDPDRPLRAGDFVCTLGFTSSDCAT | |
| | | | VDLAALRLGAVAVPLQAGASASALRPI | |
| | | | LAETAPKAVASSIELLDTVVDAVLAESA | |
| | | | PPWLIVFDYHPDIDDQRERFEAAARRL | |
| | | | AEAGAPTVLEPLAEVISRGETLGPAPLN | |
| | | | VPAPDENPLSLLIYTSGSTGTPKGAMY | |
| | | | TQSMVRGMWRGDPDDDRPAVTLNFM | |
| | | | PMSHIAGRVALIGTLVRGGLCCFTASS | |
| | | | DMSTLLEDYSLVRPTELMVVPRVCELV | |
| | | | HERFRAEVDRRAIDSADDTAIEEAVRA | |
| | | | ELREQVFGGRVQVVMCGSAPLAPEIAE | |
| | | | FMRTCLRVPVFNGYGATEAGGIVTING | |
| | | | QVQRPPVIDYKLADVPELGYYRTDSPH | |
| | | | PRGELLIKTKQLFPGYYRRPEITAEMFD | |
| | | | EDGFYRTGDIMAEVEPDHLEYVDRTKN | |
| | | | VLKLSQGEFVTVAAVQSVLTQAPLVRQ | |
| | | | IFVYGNSERSYLLAVVVPTPDAVERNA | |
| | | | GSEPALRAAIAESLRRRARDAGLASYE | |
| | | | VPRDFLIETEPFTQANGLLSDVGKTLAP | |
| | | | RLRERYGERLEQLYAELAAGQDAQLSE | |
| | | | LRRTGRDRPVLETIGKAAQALLGCAGT | |
| | | | DVRPDAHFTDLGGDSLSALTISTLLEEI | |
| | | | FGVPVPVGVVTSPATDLRALAAYVEAE | |
| | | | RASTATRPTFATVHGANATEVRATELT | |
| | | | LDRFLDADTLAATRDLPPAAETANTVLL | |
| | | | TGSTGYLGRFLCLEWMRRLDERGGRV | |
| | | | IAVVRGRDAESARERLDAAFDSGDEEL | |
| | | | ARRYAKLAADTLEVLPGDIGEPRFGLD | |
| | | | EQTWNRLATEVDLIVHPAALVNHVLPY | |
| | | | DQLFGPNVAGTAEVIRLALTSRLKPVTY | |
| | | | LSTVGVAGQASASALDEDADIRATNP | |
| | | | VRRVDDSYASGYGTSKWAGEVLLREA | |
| | | | HDLCALPVAVFRSDMILAHSAYTGQLN | |
| | | | LPDMFTRLLFSLAVTGIAPYSFYLSGAG | |
| | | | EPRPRAHYDGLPVDFTAAAVTELGERA | |
| | | | TTGFATYNTVNPHDDGVSLDVVVDWL | |
| | | | TEAGRPIERIDDYADWFVRLETALRGL | |
| | | | PENRRQYTLLPLLHAYAAPDLPIPGSVI | |
| | | | PATRFRAAVRSAGIGTDGDIPHLSAELI | |
| | | | AKYVSDLTHLRVL* | |
| Streptomyces | WP_050362911.1 | OP031620 | MGSSHHHHHHMAEPLDAAAVPAHDP | 5 |
| europaeiscabiei/ | | | GQGLAEVLASVEPGRALAEVMASVLES | |
| CAR | | | HGDRPALGERARDPETGRLLPHFDTIS | |
| (seCAR) | | | YRELWSRVRALAGRWHHDPAYPLGPG | |
| | | | DRICTLGFTSTDYATLDLACIHLGAVPV | |
| | | | PLQSNAALPRLAPIVEESGPTVLAASVD | |
| | | | RLDTAVDVVLASRTIRRLLVFDDGPGT | |
| | | | TRPSGALAAARERLAGSPVTVDTLAELI | |
| | | | DRGRDLPPPPLHTPDPGEDPLALLIYTS | |
| | | | GSTGAPKGAMYTQRLLGTAWYGFSYG | |
| | | | AADTPAISVLYLPQSHLAGRYAVMGSL | |
| | | | VKGGTGYFTAADDLSTLFEDIALVRPT | |
| | | | ELTMVPRLCDMLLQHYRSELERRSDEP | |
| | | | GDIEAAVRKAVREDFLGGRVAKAFVG | |
| | | | TAPLSAELTAFVESVLGFHLYTGYGSTE | |
| | | | AGGVLLDTVVQRPPVTDYKLVDVPELG | |
| | | | YYATDLPHPRGELLLKSHTLIPGYYRRP | |
| | | | DLTATIFDADGYYRTGDVFAETGPDRL | |
| | | | VYVDRTKDTLKLSQGEFVAVSRLETVL | |
| | | | LDSPLVQHLYLYGNSERAYLLAVVVPTP | |
| | | | AALAGSGGDTEALRPLLMESLRSVARR | |
| | | | AGLNAYEIPRGILVEPEPFSAGNGLFTE | |
| | | | SHKLLRPRLKERYGPVLELLYDQLADG | |
| | | | QDRRLRELRRTGADRPVPETVVRAAQ | |
| | | | ALLGCLSSDLRPGAHFTDLGGDSLSAV | |
| | | | SFSELLKEIFHVDVPVGVIIGPAADLAE | |
| | | | VARYITAARRPTGIRRPTFASVHGEHLT | |
| | | | EVRAGDLVPEKFLDAPTLAAAPGLPRP | |
| | | | DGDVRTVLLTGATGYLGRFLCLEWLER | |
| | | | LAPSGGRLICLVRGSDATVATRRLEAA | |
| | | | FDSGDAALLRRYRKAAAKTLEVVAGDI | |
| | | | GEPLLGLAEDTWRELAGTVDLIVHPAA | |
| | | | LVNHLLPYGELFGPNVVGTAEVIRLALT | |
| | | | ARLKPVNHVSTVAVCLGTPAETADENA | |
| | | | DIRATVPVRTIGQGYADGYATSKWAG | |
| | | | EVLLREAHERYGLPVAVFRSDMVLAHR | |
| | | | TYAGQVNVPDVLTRLLLSLVNTGIAPG | |
| | | | SFYRTDTRAHYDGLPVDFTAEAVVALG | |
| | | | ARVTEGHRTFNVLNPHDDGVCLDTFV | |
| | | | DWLIEAGHPIRRIDDHGAWLTRFTAAL | |
| | | | RALPEKHRQHSLLPLIGAWAEPDEGAP | |
| | | | GPLLPAERFHAAVRAAGVGPERDIPRV | |
| | | | SPDLIRKYVTDLRALGLLVDP* | |
| Streptomyces | WP_012382217.1 | OP031621 | MGSSHHHHHHMAEPLDAATASAHDP | 6 |
| griseus/ | | | GQGLAEALAAVEPGRALAEVMASVLE | |
| CAR | | | GHGDRPALGERAREPETGRLLPHFDTI | |
| (sgCAR) | | | SYRELWSRVRALAGRWHHDPEYPLGP | |
| | | | GDRICTLGFTSTDYATLDLACIHLGAVP | |
| | | | VPLPSNAPLPRLAPVVEESGPTVLAASV | |
| | | | DRLDTAIDVVLASSTIRRLLVFDDGPG | |
| | | | ATRPGGALAAARQRLSGSPVTVDTLA | |
| | | | GLIDRGRDLPPPPLYIPDPGEDPLALLIY | |
| | | | TSGSTGAPKGAMYTQRLLGTAWYGFS | |
| | | | YGAADTPAISVLYLPQSHLAGRYAVMG | |
| | | | SLVKGGTGYFTAADDLSTLFEDIALVR | |
| | | | PTELTMVPRLCDMLLQHYRSERDRRA | |
| | | | DEPGDIEAAVTKAVREDFLGGRVAKAF | |
| | | | VGTAPLSAELTAFVESVLGFHLYTGYG | |
| | | | STEAGGVLLDTVVQRPPVTDYKLVDVP | |
| | | | ELGYYATDLPHPRGELLLKSHTLIPGYY | |
| | | | RRPDLTAAIFDADGYYRTGDVFAETGP | |
| | | | DRLVYVDRTKDTLKLSQGEFVAVSRLE | |
| | | | TVLLDSPLVQHLYLYGNSERAYLLAVV | |
| | | | VPTPDALAGCGGDTEALRPLLMESLRS | |
| | | | VARRAGLNAYEIPRGILVEPEPFSPENG | |
| | | | LFTESHKLLRPRLKERYGPALELLYDRL | |
| | | | ADGQDRRLRELRRTGADRPVQETVLR | |
| | | | AAQALLGSPGSDLRPGAHFTDLGGDS | |
| | | | LSAVSFSELMKEIFHVDVPVGAIIGPAA | |
| | | | DLAEVARYITAARRPAGAPRPTPASVH | |
| | | | GEHRTEVRAGDLAPEKFLDAPTLAAAP | |
| | | | ALPRPDGDVRTVLLTGATGYLGRFLCL | |
| | | | EWLERLAPSGGRLVCLVRGSDATVAA | |
| | | | RRLEAAFDSGDTALLRRYRKAAGKTLD | |
| | | | VVAGDIGEPLLGLAEETWRELAGAVDL | |
| | | | IVHPAALVNHLLPYGELFGPNVVGTAE | |
| | | | AIRLALTTRLKPVNHVSTVAVCLGTPAE | |
| | | | TADENADIRAAVPVRTTGQGYADGYA | |
| | | | TSKWAGEVLLREAHERYGLPVAVFRS | |
| | | | DMVLAHRTYTGQVNVPDVLTRLLLSLV | |
| | | | ATGIAPGSFYRTDTRAHYDGLPVDFTA | |
| | | | EAVVALGAPITEGHRTFNVLNPHDDGV | |
| | | | SLDTFVDWLIEAGHPIRRIDDHGAWLT | |
| | | | RFTAALRALPEKQRQHSLLPLIGAWAE | |
| | | | PGEGAPGPLLPARRFHAAVRAAGVGPE | |
| | | | RDIPRVSPDLIRKYVTDLRALGLLAGP* | |
| Streptomyces | WP_128435011.1 | OP031619 | MGSSHHHHHHMTTGDEHSRGAGRLE | 7 |
| cyaneus/ | | | SVDAAVRDPTDGLAHSIAALMEAYAER | |
| CAR | | | PALGERAREAVTDPVSGRTALRLLPRF | |
| (scCAR) | | | TTITYAELWERAGAVAAEWTLDDQRP | |
| | | | VKPGDFVATYGFTSVDHTVLDLACLRL | |
| | | | GAVAVPLQSGAPVSRLRPVIKETGPRV | |
| | | | LAASVEVLDSAVELVLASASKPRLVVF | |
| | | | DHHPEIDDEREKFEAARRRLADAGLSC | |
| | | | IDALTAVTERGRTLGCPPVYRPAASDD | |
| | | | DPTRLLIYTSGSTGTPKGAIYTERMLTR | |
| | | | LWTGWLPAQDALSPTTINYMPLSHMA | |
| | | | ARASLYGTLGHGGTACFTAKSDMSTLF | |
| | | | EDMSLTRPTQLLLVPRVCDMLHEEYRT | |
| | | | ESARRAPEFTDTDALETAVRADLRERR | |
| | | | LGGRVRQVTCGTAPISPELKRFVESCL | |
| | | | EVPLHNGYGSTEAGPVLLDSRVQRPPV | |
| | | | IEYKLVDVPELGYFTTDVPYPRGELLLK | |
| | | | TECVTPGYYKRPEATAAVFDEDGFYRS | |
| | | | GDIMAETGPDQLVYVDRRNNVLKLAQ | |
| | | | GEFVTVSRLESVFVTSPLIRQIYVYGNS | |
| | | | ERAYLLAVIVPTDEAVRQATSHDDLRR | |
| | | | TLAESLQLMARQAELETYEIPRDFLVEP | |
| | | | EPFSVENGLLSEVRKNLRPQLRNRYGD | |
| | | | GLEALYEQLAGGRQEVLRDLREAGPG | |
| | | | QPVFEAIRRAASALLGSPAADLSPTARF | |
| | | | TDLGIDSLSALSFSRLLHDIFEVEVPVG | |
| | | | VLLSPANNLKGIADHIEAARVSGTRRP | |
| | | | SFATVHGPGSSAARAADLTLEKFIDSE | |
| | | | TLAAALRRGRPPEQSAPRTVLLTGANG | |
| | | | YLGRFMCLDWLERLATTGGRLVCIVRG | |
| | | | RDNADARRRLDAAFDSGDEELLHRYR | |
| | | | DLAARRLDVFAGDVGQGRLGLDQDM | |
| | | | WKSLTEDIDLIFHPAALVNHVLPYDQL | |
| | | | FGPNVAGTTELIRLALTGRTKPFTYLST | |
| | | | VGVATALDPSRLDEDADIREVSPVREL | |
| | | | SDAYAGGYATSKWAGEVLLREAHDAC | |
| | | | GLPVTVLRSDMILAHRRHTGQLNVPD | |
| | | | MFTRLLFSLVTTGIAPTSFYRTDDDGN | |
| | | | PRRAHYDGLPVDFVARAVNTLGTDNT | |
| | | | DGYRTYNVVNPHDDGISLDTFVDWLT | |
| | | | DAGHTIHRIDDHGTWLTRFETALRALP | |
| | | | ETQRRYSALPLLHAFRRPDEPVNGSLV | |
| | | | PAHRFEKAVQEAGLDGGGIPHLSTDLI | |
| | | | AKYVTDLRHLNLL* | |
| Mycobacteroides | WP_005082584.1 | OP031625 | MGSSHHHHHHMTETISTAAVPTTDLE | 8 |
| abscessus/ | | | EQVKRRIEQVVSNDPQLAALLPEDSVT | |
| CAR | | | EAVNEPDLPLVEVIRRLLEGYGDRPAL | |
| (mabCAR) | | | GQRAFEFVTGDDGATVIALKPEYTTVS | |
| | | | YRELWERAEAIAAAWHEQGIRDGDFV | |
| | | | AQLGFTSTDFASLDVAGLRLGTVSVPL | |
| | | | QTGASLQQRNAILEETRPAVFAASIEYL | |
| | | | DAAVDSVLATPSVRLLSVFDYHAEVDS | |
| | | | QREALEAVRARLESAGRTIVVEALAEA | |
| | | | LARGRDLPAAPLPSADPDALRLLIYTSG | |
| | | | STGTPKGAMYPQWLVANLWQKKWLT | |
| | | | DDVIPSIGVNFMPMSHLAGRLTLMGTL | |
| | | | SGGGTAYYIASSDLSTFFEDIALIRPSE | |
| | | | VLFVPRVVEMVFQRFQAELDRSLAPGE | |
| | | | SNSEIAERIKVRIREQDFGGRVLSAGS | |
| | | | GSAPLSPEMTEFMESLLQVPLRDGYGS | |
| | | | TEAGGVWRDGVLQRPPVTDYKLVDVP | |
| | | | ELGYFTTDSPHPRGELRLKSETMFPGY | |
| | | | YKRPETTADVFDDEGYYKTGDVVAELG | |
| | | | PDHLKYLDRVKNVLKLAQGEFVAVSKL | |
| | | | EAAYTGSPLVRQIFVYGNSERSFLLAVV | |
| | | | VPTPEVLERYADSPDALKPLIQDSLQQ | |
| | | | VAKDAELQSYEIPRDFIVETVPFTVESG | |
| | | | LLSDARKLLRPKLKDHYGERLEALYAEL | |
| | | | AESQNERLRQLAREAATRPVLETVTDA | |
| | | | AAALLGASSSDLAPDVRFIDLGGDSLS | |
| | | | ALSYSELLRDIFEVDVPVGVINSVANDL | |
| | | | AAIARHIEAQRTGAATQPTFASVHGKD | |
| | | | ATVITAGELTLDKFLDESLLKAAKDVQP | |
| | | | ATADVKTVLVTGGNGWLGRWLVLDW | |
| | | | LERLAPNGGKVYALIRGADAEAARARL | |
| | | | DAVYESGDPKLSAHYRQLAQQSLEVIA | |
| | | | GDFGDQDLGLSQEVWQKLAKDVDLIV | |
| | | | HSGALVNHVLPYSQLFGPNVAGTAEII | |
| | | | KLAISERLKPVTYLSTVGIADQIPVTEF | |
| | | | EEDSDVRVMSAERQINDGYANGYGNS | |
| | | | KWAGEVLLREAHDLAGLPVRVFRSDM | |
| | | | ILAHSDYHGQLNVTDVFTRSIQSLLLT | |
| | | | GVAPASFYELDADGNRQRAHYDGVPG | |
| | | | DFTAASITAIGGVNVVDGYRSFDVFNP | |
| | | | HHDGVSMDTFVDWLIDAGYKIARIDD | |
| | | | YDQWLARFELALKGLPEQQRQQSVLP | |
| | | | LLKMYEKPQPAIDGSALPTAEFSRAVH | |
| | | | EAKVGDSGEIPHVTKELILKYASDIQLL | |
| | | | GLV* | |
| Streptomyces | WP_102925360.1 | OP031623 | MGSSHHHHHHMGNTSQSNATRTQLE | 9 |
| noursei/ | | | AHRKRRIAELLSAEPGLVDLKPLPEVFE | |
| CAR | | | ATAERGLRLSEIIKRTLAGYADRPALGE | |
| (snCAR) | | | RAKEFVPVAGRTEMRLLPHFRTLTYSEL | |
| | | | WLRLSAVAADWYHHPEHPLRAGDFVA | |
| | | | VLGFAGTDYTTVDLACSQVGAVAVPLH | |
| | | | SNSTVAHIRPLVTEAAPRIFATSAERLE | |
| | | | MVAEALTGDTSVRRLIVFDYHPELDEH | |
| | | | REHMAAARRRLAEAGSPISLELLSHVI | |
| | | | DCGRTSPPAPDLPQEDRAGETLSALIY | |
| | | | TSGSTGSPKGVMYAESTVSLMWRPN | |
| | | | WDPSYQFPVITANFFPQSHLVARRILA | |
| | | | TTLARGGTAYFVASSDLSTFLEDLALIR | |
| | | | PTDLTVVPRVCEMLFQGYQSRLDQRAP | |
| | | | RAELREADYTEVLAHFRDEVFGGRIVR | |
| | | | ILCGSAPLAPELVAFLESCLEVPFHNGY | |
| | | | GTTETGFLMLDHRVQRPPVVDYKLVD | |
| | | | VPELGYLTTDSPHPRGELLVKTRLITSG | |
| | | | YYQRPELTAEVFDEEGFYRTGDIMAEIE | |
| | | | PDNLVYIDRRNNVLKLSQGEFVALARL | |
| | | | ESVFATSPLVHQIYLYGNSSRAYLLAVV | |
| | | | VPTHPAIQQASDRAALTSTISESLQRIA | |
| | | | KEAKLNPYEIPRDLLIETEPFSQDNGLL | |
| | | | SDSTKHIRPRLRERYGEQLERRYAELA | |
| | | | EGQANELRGLRHDKSRPVVETVTRAV | |
| | | | QALLGMPEGELNPSTRFTDLGGDSLSA | |
| | | | LSLSHLLRETFDAEVPVGTLIGPANSLR | |
| | | | QIADHIEQLRSPSTERPTFTRVHGEHA | |
| | | | TAARAEDLTLDTFIDQDTLTAATSLPQP | |
| | | | TDTPGTVLLTGANGYLGRFLCLEWLER | |
| | | | LAPIGGTLICLVRGADNTAARRRLEQA | |
| | | | LDSGDAELQRRFGELATNGTLQVLAG | |
| | | | DIGMANLGLTDQTWQQLAATVDLIVH | |
| | | | PAALVNHVLPYRQLFGPNVVGTAELIR | |
| | | | MALTTRLKPISYLSTVAVASHGAATLD | |
| | | | EDIDIRVASPARPIDERHAGGYALSKW | |
| | | | AGEVLLREAHDLCGLPVTVFRSDMILA | |
| | | | HGRYTGQLNVPDTFTRLMLSLIATGIA | |
| | | | PQSFYRTEDSGQQPRAHYDGLPGGFV | |
| | | | ASSVATLGDGTAGYRTFNVVNPHDDG | |
| | | | ISLDTVVDWLADSGTPIHRIEDYQEW | |
| | | | HERFEAALRALPEKQRQHSLLPIVHAY | |
| | | | REPATPLAGSVVPATRFQEAVRAAGIG | |
| | | | SEKEIPHLSRPLIEKYLTDLRHLGLV* | |
| Mycolicibacterium | AFP42026.1 | OP031624 | MGSSHHHHHHMHQLTVTGMNICEVQ | 10 |
| smegmatis/ | | | RLFPRMTSDVHDATDGVTETALDDEQ | |
| CAR | | | STRRIAELYATDPEFAAAAPLPAVVDAA | |
| (msCAR) | | | HKPGLRLAEILQTLFTGYGDRPALGYR | |
| | | | ARELATDEGGRTVTRLLPRFDTLTYAQ | |
| | | | VWSRVQAVAAALRHNFAQPIYPGDAV | |
| | | | ATIGFASPDYLTLDLVCAYLGLVSVPLQ | |
| | | | HNAPVSRLAPILAEVEPRILTVSAEYLD | |
| | | | LAVESVRDVNSVSQLVVFDHHPEVDD | |
| | | | HRDALARAREQLAGKGIAVTTLDAIAD | |
| | | | EGAGLPAEPIYTADHDQRLAMILYTSG | |
| | | | STGAPKGAMYTEAMVARLWTMSFITG | |
| | | | DPTPVINVNFMPLNHLGGRIPISTAVQ | |
| | | | NGGTSYFVPESDMSTLFEDLALVRPTE | |
| | | | LGLVPRVADMLYQHHLATVDRLVTQG | |
| | | | ADELTAEKQAGAELREQVLGGRVITGF | |
| | | | VSTAPLAAEMRAFLDITLGAHIVDGYG | |
| | | | LTETGAVTRDGVIVRPPVIDYKLIDVPE | |
| | | | LGYFSTDKPYPRGELLVRSQTLTPGYYK | |
| | | | RPEVTASVFDRDGYYHTGDVMAETAP | |
| | | | DHLVYVDRRNNVLKLAQGEFVAVANL | |
| | | | EAVFSGAALVRQIFVYGNSERSFLLAV | |
| | | | VVPTPEALEQYDPAALKAALADSLQRT | |
| | | | ARDAELQSYEVPADFIVETEPFSAANG | |
| | | | LLSGVGKLLRPNLKDRYGQRLEQMYA | |
| | | | DIAATQANQLRELRRAAATQPVIDTLT | |
| | | | QAAATILGTGSEVASDAHFTDLGGDSL | |
| | | | SALTLSNLLSDFFGFEVPVGTIVNPATN | |
| | | | LAQLAQHIEAQRTAGDRRPSFTTVHGA | |
| | | | DATEIRASELTLDKFIDAETLRAAPGLP | |
| | | | KVTTEPRTVLLSGANGWLGRFLTLQW | |
| | | | LERLAPVGGTLITIVRGRDDAAARARL | |
| | | | TQAYDTDPELSRRFAELADRHLRVVAG | |
| | | | DIGDPNLGLTPEIWHRLAAEVDLVVHP | |
| | | | AALVNHVLPYRQLFGPNVVGTAEVIKL | |
| | | | ALTERIKPVTYLSTVSVAMGIPDFEEDG | |
| | | | DIRTVSPVRPLDGGYANGYGNSKWAG | |
| | | | EVLLREAHDLCGLPVATFRSDMILAHP | |
| | | | RYRGQVNVPDMFTRLLLSLLITGVAPR | |
| | | | SFYIGDGERPRAHYPGLTVDFVAEAVT | |
| | | | TLGAQQREGYVSYDVMNPHDDGISLD | |
| | | | VFVDWLIRAGHPIDRVDDYDDWVRRF | |
| | | | ETALTALPEKRRAQTVLPLLHAFRAPQA | |
| | | | PLRGAPEPTEVFHAAVRTAKVGPGDIP | |
| | | | HLDEALIDKYIRDLREFGLI* | |
| Cafeteria | KAA0160565.1 | OP031628 | MGSSHHHHHHMADSLPLLAPQDSYQ | 11 |
| roenbergensis/ | | | SGLAACVSEREESILTRVAAKPEMPRV | |
| CAR | | | AYAMVFPEEPPFAPHPNDPDIEALLAG | |
| (crCAR) | | | GGLEVLTKSFLLGRLQWTPLPCVCRPA | |
| | | | EAGAARRTFDASLPSSALFGDGGAVFV | |
| | | | RVAHMARRRWRHNWVAHFAASDSTA | |
| | | | ELAALHPHLPTISGGQRSHVSNEAMDT | |
| | | | RATVALCEARGSHPDAWPAAGSPSRR | |
| | | | WCCGLELPPRTRSVCVVVGVLDAERS | |
| | | | VPLVRSTYVPGLQWPMQSSLAKEADS | |
| | | | TESREAFQAGKPYWEAPIAPAPTDGSV | |
| | | | ASPTEIEPRPWYDFEVSSRSASADVG | |
| | | | WPVFVGRVKVTIRREQPDGAPPIEGPV | |
| | | | ELEVIGASLANFRDFQTRRSLIPRSFW | |
| | | | RAKLVIPSGQTSAFCLPRRCAQIVRVV | |
| | | | LASPSPASLWVHDIDVRIASCWGSTP | |
| | | | QDDMAASLDLFGPRPALGMRFPIAGG | |
| | | | AAAAAGPAARVADPAAASPSPPSASQ | |
| | | | GAGKDDALSGVLEEAGTGSEDKLGPY | |
| | | | EWLSYKDFATFANRTSSGLEGSLLGLL | |
| | | | GDGDATRRLPPPPADSEAAGSAAAGS | |
| | | | AAADSAAAGGSASSAGSGADSSDRV | |
| | | | FMGICASNRMEWLAAELAGQHGSFVH | |
| | | | VPIMTTASQEVVDHVVSQTGMMVAVA | |
| | | | EPGEPLARLLSTQKRLGLPELLVVIDTT | |
| | | | HFGARDPACPDVTADPAFAPAAQLVD | |
| | | | GSARKAGRAFALARLSDIVEEGRRRLV | |
| | | | AHGHESEAGERLLRFPMPTQRMRDDA | |
| | | | KAAFAECSEERQTQLALLRHPDRIASP | |
| | | | GWASSRAWVLTVDTSKPPGGAGRRV | |
| | | | ALGQGPGLSDLACVVYTSGSTGLPKG | |
| | | | VQRSFQSNMEALHEFFAPTVAVHFSV | |
| | | | QPLAHLSEAHSLPSVLVSGGQVGFAT | |
| | | | GGRHGVYGDVAELEPTFLNTVPAFFNR | |
| | | | LHALFSAALAVKAAGAPEAARAAIRQA | |
| | | | LLKEFQVALGGRLQSIGIGSAPVTKGV | |
| | | | LDWMTVCFSQAQVGEGYGSTECGTIS | |
| | | | NGNKIAEGVEFRLDDIPELGYLTSGSP | |
| | | | PRGEILVRTKWSSEGYFRNPKATSDAL | |
| | | | TDDGFFRTGDVGEQLEDGRVMIIGRR | |
| | | | KFVTKLANGEFVSLERIETVLSKSDLVD | |
| | | | QVFVDADGSEYGVVAVVVAVPTALAA | |
| | | | AVSARPAGKQDADALRKLARSAEAAA | |
| | | | ALKADLARVAAESGLQPFEAPKAVFLE | |
| | | | TELRFTAENGLLTGSNKTSRSGLRRKY | |
| | | | SAIVKALYSVAGANPGADELLKRAAEL | |
| | | | GAAGGDSDGPGAAAAGGAAAAEPTS | |
| | | | VEDGVRATVSRLVFETLGVSDGDLVA | |
| | | | ALGSDSLRVSSLSAMVGSALGVDLAA | |
| | | | AANRASTVRELVDEVTALVLESRGLAR | |
| | | | APSAAAGAGGKAPLHGAAETATADME | |
| | | | ALGGWVASAMGGVPAWEAERGVDIA | |
| | | | LATDVPKPGSQSSIASAEAETVLLTGA | |
| | | | TGFLGVHLLVQLLQQSDKRVVALARPS | |
| | | | RGTSGAQGIRQARVGAESEAEVDGGG | |
| | | | SSAAAPARQTSAGRTGDELAEQRVVA | |
| | | | AVKETRCDLPAGWRGRLTVLASDIAKP | |
| | | | MLGLPAARWRELRDDPSLCVVHCAAR | |
| | | | VNWLMRYEQLRASNVLGTLEVIRLCAA | |
| | | | GASRHPLHFVSTISVGDTARGAAESD | |
| | | | RMPFDRVVAGLQAGQGGYGPSKWLA | |
| | | | EAAVVRAGEHGTAGSGLFVSVHRPGM | |
| | | | ITAHSQTGHSNEQDFVNRYIAACASLG | |
| | | | VALGPGLPADARLDMTPVDFVAGGVT | |
| | | | RIALAHGAKASAPDAGWRRSGSCFQY | |
| | | | VNADGSPRFADIGAWIREAGYNCEPR | |
| | | | GYADFRDELHSRAAEGHALTPLLDFFP | |
| | | | PGDFPESLGAVHGGDTLTRAALEDLAE | |
| | | | AGTADPGDAALVPAPPKVTAVVIRRTL | |
| | | | ASLAVRGFVPAPDAAAPSVPETTLRPA | |
| | | | RQVLEAASREAAESHRGRTNREVLALL | |
| | | | TQAVQDDAEVDLSPLLRSLVDAYG* | |
| Mycobacterium | WP_003872682.1 | OP031609 | MGSSHHHHHHMSTATHDERLDRRVH | 12 |
| avium/CAR | | | ELIATDPQFAAAQPDPAITAALEQPGLR | |
| (mavCAR) | | | LPQIIRTVLDGYADRPALGQRVVEFVT | |
| | | | DAKTGRTSAQLLPRFETITYGEVAQRV | |
| | | | SALGRALSDDAVHPGDRVCVLGFNSV | |
| | | | DYATIDMALGAIGAVSVPLQTSAAISS | |
| | | | LQPIVAETEPTLIASSVNQLSDAVQLIT | |
| | | | GAEQAPTRLVVFDYHPQVDDQREAVQ | |
| | | | DAAARLSGTGVAVQTLAELLERGKDLP | |
| | | | AVAEPPADEDSLALLIYTSGSTGAPKG | |
| | | | AMYPQSNVGKMWRRGSKNWFGESAA | |
| | | | SITLNFMPMSHVMGRSILYGTLGNGGT | |
| | | | AYFAARSDLSTLLEDLELVRPTELNFVP | |
| | | | RIWETLYGEFQRQVERRLSEAGDAGE | |
| | | | RRAVEAEVLAEQRQYLLGGRFTFAMTG | |
| | | | SAPISPELRNWVESLLEMHLMDGYGS | |
| | | | TEAGMVLFDGEIQRPPVVDYKLVDVPD | |
| | | | LGYFSTDRPHPRGELLLRTENMFPGYY | |
| | | | KRAETTAGVFDEDGYYRTGDVFAEIAP | |
| | | | DRLVYVDRRNNVLKLAQGEFVTLAKLE | |
| | | | AVFGNSPLIRQIYVYGNSAQPYLLAVV | |
| | | | VPTEEALASGDPETLKPKIADSLQQVA | |
| | | | KEAGLQSYEVPRDFIIETTPFSLENGLL | |
| | | | TGIRKLAWPKLKQHYGERLEQMYADL | |
| | | | AAGQADELAELRRNGAQAPVLQTVSR | |
| | | | AAGAMLGSAASDLSPDAHFTDLGGDS | |
| | | | LSALTFGNLLREIFDVDVPVGVIVSPAN | |
| | | | DLAAIASYIEAERQGSKRPTFASVHGR | |
| | | | DATVVRAADLTLDKFLDADTLASAPNL | |
| | | | PKPATEVRTVLLTGATGFLGRYLALEW | |
| | | | LERMDMVDGKVIALVRARSDEEARAR | |
| | | | LDKTFDSGDPKLLAHYQQLAADHLEVI | |
| | | | AGDKGEANLGLRQDVWQRLADTVDVI | |
| | | | VDPAALVNHVLPYSELFGPNALGTAELI | |
| | | | RLALTSKQKPYTYVSTIGVGDQIEPGK | |
| | | | FVENADIRQMSATRAINDSYANGYGN | |
| | | | SKWAGEVLLREAHDLCGLPVAVFRCD | |
| | | | MILADTTYAGQLNLPDMFTRLMLSLVA | |
| | | | TGIAPGSFYELDADGNRQRAHYDGLP | |
| | | | VEFIAAAISTLGSQITDSDTGFQTYHV | |
| | | | MNPYDDGIGLDEYVDWLVDAGYSIER | |
| | | | IADYSEWLRRFETSLRALPDRQRQYSL | |
| | | | LPLLHNYRTPEKPINGSIAPTDVFRAAV | |
| | | | QEAKIGPDKDIPHVSPPVIVKYITDLQL | |
| | | | LGLL* | |
| Trichoderma | XP_006964071.1 | OP031611 | MGSSHHHHHHMRSFVKANVDFSSAE | 13 |
| reesei/CAR | | | RKEDYIHSLPELVDFNAVQNPNHLLCI | |
| (trCAR) | | | QARSNAPWVKITNAQFKVAIDQCATW | |
| | | | IAENVKLPKARTKHDLTGRLPVALLME | |
| | | | SDFGLLVHQFALVSMGIPPLVLSARLSP | |
| | | | EAIFHLLRSTEASSLIVSQRVAMITKGA | |
| | | | FGNVKTSDFHVAQPYSTFCNVPADKS | |
| | | | VRKQSVYPDNIDANIVLLHSSGTTGLP | |
| | | | KPIALSHRQLMFSVSHGDFETEEEAQG | |
| | | | IVISTLPLFHGFGLLAPGLSMAIGKTVC | |
| | | | FPASDEVPDAQSIVDLINMSGATGMLT | |
| | | | VPFLLENMAALPNGTGLRALAKLDFVG | |
| | | | TGGSALSADFGVSASAAGVKLLNLYGT | |
| | | | TETGPLTKTFAPKSGYDWKYFRLRQD | |
| | | | MLFKVTELPPVDGEKRFRLTVFPFGAD | |
| | | | KPFEIADQLIRSEKFPETDFAAVGRDD | |
| | | | DVVVLATGEKVNPLLLETALTDSGLVK | |
| | | | SAIVFGENQFQIGVVVEPATPLNPDQK | |
| | | | EEFRKKIWPIIVRVGERMDTTARIYSPN | |
| | | | AVIVVPSSVTIPRTDKGSIARKEVFQLL | |
| | | | EKEISQVYEDLENGSIEETPLDYDKLEQ | |
| | | | ELKGLIQKRLKLRVHPGKWTVDDNLF | |
| | | | HLGLDSLQATTLRRILLSAASKTPPDVI | |
| | | | GKDFIYVNPSVKAIANALRPANGPIGT | |
| | | | ESASVAQEVDDYAQQYSIKGFEVQDI | |
| | | | VPKASPKLIRGAVVLLTGSSGGLGSHA | |
| | | | LGKLAESTQVAKIVCLQRKRPGTVINPI | |
| | | | PGAAKVDRASIEAKGIKLTDDQWAKIT | |
| | | | ALEIDPTIDNLGLPAMVMGMVSKTVTH | |
| | | | ILHAAWPMDFHMRLPSFGYQFSYLKNL | |
| | | | LRIAVQAPQKVRFLFVSSISALAKLGLI | |
| | | | TPGRPIPEEPLDVESAACGIGYADAKLV | |
| | | | CEKILEEAASLYNSNVEVVIARCGQLS | |
| | | | GARKTGAWNVSEQIPMLIRTSQGLGIL | |
| | | | PILEGTVSWIPVDDAAATVAELLFAPD | |
| | | | APGLVTHVENPVRQSWSEVFQIIGNEL | |
| | | | RITKTLSFDDWLGEVTSTAERDVEDYP | |
| | | | VRKLYEFFKLYFRIASSGAVVMGTDMS | |
| | | | RKNSATLRCLKALDRGTIAGYVRYWR | |
| | | | SVGYLRQ* | |
| Neurospora | XP_955820.1 | OP031610 | MGSSHHHHHHMSQQQNPPYGRRLIL | 14 |
| crassa/CAR | | | DIIKERALNEPNREWVSVPRSSDPKDG | |
| (ncCAR) | | | WKILTYLDAYNGINRVAHKLTQVCGAA | |
| | | | APGSFPTVAYIGPNDVRYLVFALGAVK | |
| | | | AGYKALFISTRNSAEAQVNLFELTNCN | |
| | | | VLVFDQSYKATVQPWLHEREMTAILAL | |
| | | | PADEWFPADQEDFPYNKTFEEAEWDP | |
| | | | LMVLHTSGSTGFPKPIVARQGMLAVA | |
| | | | DQFHNLPPREDGKLMWIVEMSKRAKR | |
| | | | LMHPMPLFHAAGMYISMLMIHYWDTP | |
| | | | GALGIGERPLSSDLVLDYIEYADVEGM | |
| | | | ILPPAILEELSRDEKAIQSLQKLNFVSF | |
| | | | GGGNLAPEAGDRLVENNVTLCNLISAT | |
| | | | EFTPFPFYWQYDQKLWRYFNFDTDLFG | |
| | | | IDWRLHDGESTYEQVIVRKDKHPGLQ | |
| | | | GFFYTFPDSSEYSTKDLYKRHPTHEDF | |
| | | | WIYQGRADNIIVFSNGEKLNPITIEETL | |
| | | | QGHPKVMGAVVVGTNRFQPALIIEPVE | |
| | | | HPETEEGRKALLDEIWPTVVRVNKETV | |
| | | | AHGQIGRQYMALSTPGKPFLRAGKGT | |
| | | | VLRPGTINMYKAEIDKIYEDAEKGVAT | |
| | | | DEVPKLDLSSSDALIVSIEKLFETSLNA | |
| | | | PKLEADTDFFTAGVDSMQVITASRLIR | |
| | | | AGLAAAGVNIEASALATRVIYGNPTPK | |
| | | | RLADYLLSIVNKDSNQGTLDNEHHVM | |
| | | | EALVEKYTRDLPTPKQNKPAPADEGQV | |
| | | | VVITGTTGGIGSYLIDICSSSSRVSKII | |
| | | | CLNRSEDGKARQTASSSGRGLSTDFS | |
| | | | KCEFYHADMSRADLGLGPEVYSRLLSE | |
| | | | VDRVIHNQWPVNFNIAVESFEPHIRGC | |
| | | | RNLVDFSYKADKNVPIVFVSSIGTVDR | |
| | | | WHDEDRIVPEASLDDLSLAAGGYGQS | |
| | | | KLVSSLIFDKAAEVSGVPTEVVRVGQV | |
| | | | AGPSSEKGYWNKQEWLPSIVASSAYL | |
| | | | GVLPDSLGQMTTIDWTPIEAIAKLLLEV | |
| | | | SGVIDNVPLDKINGYFHGVNPERTSW | |
| | | | SALAPAVQEYYGDRIQKIVPLDEWLEA | |
| | | | LEKSQEKAEDVTRNPGIKLIDTYRTWS | |
| | | | EGYKKGTKFVPLDMTRTKEYSKTMRE | |
| | | | MHAVTPELMKNWCRQWNF* | |
| Segniliparus | WP_013138593.1 | OP031608 | MGSSHHHHHHMTQSHTQGPQASAAH | 15 |
| rotundus/CAR | | | SRLARRAAELLATDPQAAATLPDPEVV | |
| (srCAR) | | | RQATRPGLRLAERVDAILSGYADRPAL | |
| | | | GQRSFQTVKDPITGRSSVELLPTFDTIT | |
| | | | YRELRERATAIASDLAHHPQAPAKPGD | |
| | | | FLASIGFISVDYVAIDIAGVFAGLTAVP | |
| | | | LQTGATLATLTAITAETAPTLFAASIEHL | |
| | | | PTAVDAVLATPSVRRLLVFDYRAGSDE | |
| | | | DREAVEAAKRKIADAGSSVLVDVLDEV | |
| | | | IARGKSAPKAPLPPATDAGDDSLSLLIY | |
| | | | TSGSTGTPKGAMYPERNVAHFWGGV | |
| | | | WAAAFDEDAAPPVPAINITFLPLSHVA | |
| | | | SRLSLMPTLARGGLMHFVAKSDLSTLF | |
| | | | EDLKLARPTNLFLVPRVVEMLYQHYQS | |
| | | | ELDRRGVQDGTREAEAVKDDLRTGLL | |
| | | | GGRILTAGFGSAPLSAELAGFIESLLQI | |
| | | | HLVDGYGSTEAGPVWRDGYLVKPPVT | |
| | | | DYKLIDVPELGYFSTDSPHPRGELAIKT | |
| | | | QTILPGYYKRPETTAEVFDEDGFYLTG | |
| | | | DVVAQIGPEQFAYVDRRKNVLKLSQG | |
| | | | EFVTLAKLEAAYSSSPLVRQLFVYGSSE | |
| | | | RSYLLAVIVPTPDALKKFGVGEAAKAAL | |
| | | | GESLQKIARDEGLQSYEVPRDFIIETDP | |
| | | | FTVENGLLSDARKSLRPKLKEHYGERL | |
| | | | EAMYKELADGQANELRDIRRGVQQRP | |
| | | | TLETVRRAAAAMLGASAAEIKPDAHFT | |
| | | | DLGGDSLSALTFSNFLHDLFEVDVPVG | |
| | | | VIVSAANTLGSVAEHIDAQLAGGRARP | |
| | | | TFATVHGKGSTTIKASDLTLDKFIDEQ | |
| | | | TLEAAKHLPKPADPPRTVLLTGANGWL | |
| | | | GRFLALEWLERLAPAGGKLITIVRGKD | |
| | | | AAQAKARLDAAYESGDPKLAGHYQDL | |
| | | | AATTLEVLAGDFSEPRLGLDEATWNRL | |
| | | | ADEVDFISHPGALVNHVLPYNQLFGPN | |
| | | | VAGVAEIIKLAITTRIKPVTYLSTVAVAA | |
| | | | GVEPSALDEDGDIRTVSAERSVDEGY | |
| | | | ANGYGNSKWGGEVLLREAHDRTGLPV | |
| | | | RVFRSDMILAHQKYTGQVNATDQFTR | |
| | | | LVQSLLATGLAPKSFYELDAQGNRQRA | |
| | | | HYDGIPVDFTAESITTLGGDGLEGYRS | |
| | | | YNVFNPHRDGVGLDEFVDWLIEAGHPI | |
| | | | TRIDDYDQWLSRFETSLRGLPESKRQA | |
| | | | SVLPLLHAFARPGPAVDGSPFRNTVFR | |
| | | | TDVQKAKIGAEHDIPHLGKALVLKYAD | |
| | | | DIKQLGLL* | |
| Nocardia | Q6RKB1.1 | OP031607 | MGSSHHHHHHMAVDSPDERLQRRIA | 16 |
| iowensis/CAR | | | QLFAEDEQVKAARPLEAVSAAVSAPG | |
| (niCAR) | | | MRLAQIAATVMAGYADRPAAGQRAFE | |
| | | | LNTDDATGRTSLRLLPRFETITYRELW | |
| | | | QRVGEVAAAWHHDPENPLRAGDFVAL | |
| | | | LGFTSIDYATLDLADIHLGAVTVPLQAS | |
| | | | AAVSQLIAILTETSPRLLASTPEHLDAA | |
| | | | VECLLAGTTPERLVVFDYHPEDDDQRA | |
| | | | AFESARRRLADAGSLVIVETLDAVRAR | |
| | | | GRDLPAAPLFVPDTDDDPLALLIYTSGS | |
| | | | TGTPKGAMYTNRLAATMWQGNSMLQ | |
| | | | GNSQRVGINLNYMPMSHIAGRISLFGV | |
| | | | LARGGTAYFAAKSDMSTLFEDIGLVRP | |
| | | | TEIFFVPRVCDMVFQRYQSELDRRSVA | |
| | | | GADLDTLDREVKADLRQNYLGGRFLV | |
| | | | AVVGSAPLAAEMKTFMESVLDLPLHDG | |
| | | | YGSTEAGASVLLDNQIQRPPVLDYKLV | |
| | | | DVPELGYFRTDRPHPRGELLLKAETTIP | |
| | | | GYYKRPEVTAEIFDEDGFYKTGDIVAEL | |
| | | | EHDRLVYVDRRNNVLKLSQGEFVTVA | |
| | | | HLEAVFASSPLIRQIFIYGSSERSYLLA | |
| | | | VIVPTDDALRGRDTATLKSALAESIQRI | |
| | | | AKDANLQPYEIPRDFLIETEPFTIANGLL | |
| | | | SGIAKLLRPNLKERYGAQLEQMYTDLA | |
| | | | TGQADELLALRREAADLPVLETVSRAA | |
| | | | KAMLGVASADMRPDAHFTDLGGDSLS | |
| | | | ALSFSNLLHEIFGVEVPVGVVVSPANEL | |
| | | | RDLANYIEAERNSGAKRPTFTSVHGGG | |
| | | | SEIRAADLTLDKFIDARTLAAADSIPHA | |
| | | | PVPAQTVLLTGANGYLGRFLCLEWLER | |
| | | | LDKTGGTLICVVRGSDAAAARKRLDS | |
| | | | AFDSGDPGLLEHYQQLAARTLEVLAGD | |
| | | | IGDPNLGLDDATWQRLAETVDLIVHPA | |
| | | | ALVNHVLPYTQLFGPNVVGTAEIVRLAI | |
| | | | TARRKPVTYLSTVGVADQVDPAEYQE | |
| | | | DSDVREMSAVRVVRESYANGYGNSK | |
| | | | WAGEVLLREAHDLCGLPVAVFRSDMIL | |
| | | | AHSRYAGQLNVQDVFTRLILSLVATGI | |
| | | | APYSFYRTDADGNRQRAHYDGLPADF | |
| | | | TAAAITALGIQATEGFRTYDVLNPYDD | |
| | | | GISLDEFVDWLVESGHPIQRITDYSD | |
| | | | WFHRFETAIRALPEKQRQASVLPLLDA | |
| | | | YRNPCPAVRGAILPAKEFQAAVQTAKI | |
| | | | GPEQDIPHLSAPLIDKYVSDLELLQLL* | |
| Mycobacterium | WP_012393886.1 | OP031612 | MGSSHHHHHHMSPITREERLERRIQD | 17 |
| marinum/CAR | | | LYANDPQFAAAKPATAITAAIERPGLPL | |
| (mmCAR) | | | PQIIETVMTGYADRPALAQRSVEFVTD | |
| | | | AGTGHTTLRLLPHFETISYGELWDRIS | |
| | | | ALADVLSTEQTVKPGDRVCLLGFNSVD | |
| | | | YATIDMTLARLGAVAVPLQTSAAITQL | |
| | | | QPIVAETQPTMIAASVDALADATELAL | |
| | | | SGQTATRVLVFDHHRQVDAHRAAVES | |
| | | | ARERLAGSAVVETLAEAIARGDVPRGA | |
| | | | SAGSAPGTDVSDDSLALLIYTSGSTGA | |
| | | | PKGAMYPRRNVATFWRKRTWFEGGYE | |
| | | | PSITLNFMPMSHVMGRQILYGTLCNGG | |
| | | | TAYFVAKSDLSTLFEDLALVRPTELTFV | |
| | | | PRVWDMVFDEFQSEVDRRLVDGADR | |
| | | | VALEAQVKAEIRNDVLGGRYTSALTGS | |
| | | | APISDEMKAWVEELLDMHLVEGYGST | |
| | | | EAGMILIDGAIRRPAVLDYKLVDVPDL | |
| | | | GYFLTDRPHPRGELLVKTDSLFPGYYQ | |
| | | | RAEVTADVFDADGFYRTGDIMAEVGP | |
| | | | EQFVYLDRRNNVLKLSQGEFVTVSKLE | |
| | | | AVFGDSPLVRQIYIYGNSARAYLLAVIV | |
| | | | PTQEALDAVPVEELKARLGDSLQEVAK | |
| | | | AAGLQSYEIPRDFIIETTPWTLENGLLT | |
| | | | GIRKLARPQLKKHYGELLEQIYTDLAH | |
| | | | GQADELRSLRQSGADAPVLVTVCRAA | |
| | | | AALLGGSASDVQPDAHFTDLGGDSLS | |
| | | | ALSFTNLLHEIFDIEVPVGVIVSPANDL | |
| | | | QALADYVEAARKPGSSRPTFASVHGA | |
| | | | SNGQVTEVHAGDLSLDKFIDAATLAEA | |
| | | | PRLPAANTQVRTVLLTGATGFLGRYLAL | |
| | | | EWLERMDLVDGKLICLVRAKSDTEAR | |
| | | | ARLDKTFDSGDPELLAHYRALAGDHLE | |
| | | | VLAGDKGEADLGLDRQTWQRLADTV | |
| | | | DLIVDPAALVNHVLPYSQLFGPNALGT | |
| | | | AELLRLALTSKIKPYSYTSTIGVADQIPP | |
| | | | SAFTEDADIRVISATRAVDDSYANGYS | |
| | | | NSKWAGEVLLREAHDLCGLPVAVFRC | |
| | | | DMILADTTWAGQLNVPDMFTRMILSL | |
| | | | AATGIAPGSFYELAADGARQRAHYDGL | |
| | | | PVEFIAEAISTLGAQSQDGFHTYHVMN | |
| | | | PYDDGIGLDEFVDWLNESGCPIQRIAD | |
| | | | YGDWLQRFETALRALPDRQRHSSLLPL | |
| | | | LHNYRQPERPVRGSIAPTDRFRAAVQE | |
| | | | AKIGPDKDIPHVGAPIIVKYVSDLRLLG | |
| | | | LL* | |
| Chromobacterium | WP_011135573.1 | N/A | MGSSHHHHHHSQDPNSMQKORTTS | 18 |
| violaceum/ | | | QWRELDAAHHLHPFTDTASLNQAGAR | |
| w-transaminase | | | VMTRGEGVYLWDSEGNKIIDGMAGL | |
| (cvTA) | | | WCVNVGYGRKDFAEAARRQMEELPFY | |
| | | | NTFFKTTHPAVVELSSLLAEVTPAGFDR | |
| | | | VFYTNSGSESVDTMIRMVRRYWDVQG | |
| | | | KPEKKTLIGRWNGYHGSTIGGASLGG | |
| | | | MKYMHEQGDLPIPGMAHIEQPWWYK | |
| | | | HGKDMTPDEFGVVAARWLEEKILEIGA | |
| | | | DKVAAFVGEPIQGAGGVIVPPATYWPE | |
| | | | IERICRKYDVLLVADEVICGFGRTGEW | |
| | | | FGHQHFGFQPDLFTAAKGLSSGYLPIG | |
| | | | AVFVGKRVAEGLIAGGDFNHGFTYSG | |
| | | | HPVCAAVAHANVAALRDEGIVQRVKD | |
| | | | DIGPYMQKRWRETFSRFEHVDDVRGV | |
| | | | GMVQAFTLVKNKAKRELFPDFGEIGTL | |
| | | | CRDIFFRNNLIMRACGDHIVSAPPLVM | |
| | | | TRAEVDEMLAVAERCLEEFEQTLKARG | |
| | | | LA* | |

˜Please note that all enzymes listed in the table above start with the amino acids MGSS followed by a hexahistidine purification tag HHHHHH, before the amino acid sequence of the functional enzyme begins. The “*” denotes the position of the stop codon.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for preparing an amine product in a single reaction mixture, comprising incubating a biomass-derived or plastic-derived aryl or heterocyclic carboxylic acid, a carboxylic acid reductase (CAR), and a ω-transaminase (TA) in the single reaction mixture, reducing the biomass-derived or plastic-derived aryl or heterocyclic carboxylic acid to an aryl or heterocyclic aldehyde, and transferring an amine to the aryl or heterocyclic aldehyde, whereby the amine product is produced in the single reaction mixture.

2. The method of claim 1, wherein the biomass-derived aryl carboxylic acid is a guaiacol and syringol 4-substituted carboxylic acid of Formula I 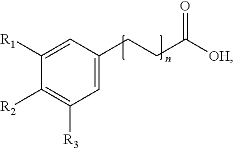
the aryl aldehyde is of Formula II 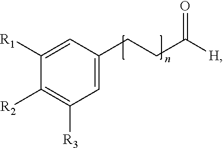
and the amine product is of Formula III

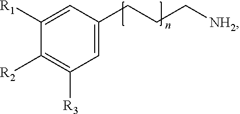

3. The method of claim 2, wherein R₁, R₂, R₃ and n in Formula I, Formula II and Formula III are selected from the group consisting of:
(a) R₁═H, R₂═OH, R₃═OCH₃, and n=0;
(b) R₁═OCH₃, R₂═OH, R₃═OCH₃, and n=0;
(c) R₁═H, R₂═H, R₃═H, and n=1 (unsaturated);
(d) R₁═H, R₂═OH, R₃═OCH₃, and n=1 (unsaturated); and
(e) R₁═H, R₂═OH, R₃═OCH₃, and n=1 (saturated).

4. The method of claim 2, wherein R₁, R₂, R₃ and n in Formula I, Formula II and Formula III are selected from the group consisting of:
(a) R₁═OCH₃, R₂═OH, R₃═OCH₃, and n=0;
(b) R₁═H, R₂═OH, R₃═OCH₃, and n=1 (unsaturated); and
(c) R₁═H, R₂═OH, R₃═OCH₃, and n=1 (saturated).

5. The method of claim 1, wherein the biomass-derived aryl carboxylic acid is of Formula IV 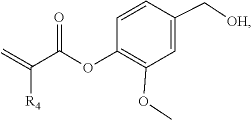
the aryl aldehyde is of Formula V 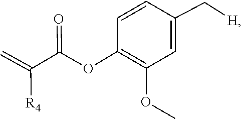
and the amine product is of Formula VI

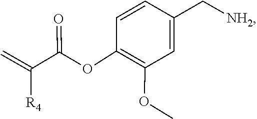

6. The method of claim 5, wherein R₄ in Formula IV, Formula V and Formula VI is H or CH₃.

7. The method of claim 1, wherein the biomass-derived aryl carboxylic acid is a furan carboxylic acid.

8. The method of claim 1, wherein the plastic-derived aryl carboxylic acid is a polyethylene terephthalate (PET)-derived aryl carboxylic acid.

9. The method of claim 8, wherein the PET-derived aryl carboxylic acid is selected from the group consisting of terephthalic acid (TPA), mono-(2-hydroxyethyl)-terephthalic acid (MHET) and monomethyl terephthalate (mmTPA).

10. The method of claim 8, wherein the PET-derived aryl carboxylic acid is terephthalic acid (TPA) and the amine product is para-xylylenediamine (pXYL).

11. The method of claim 10, further comprising producing an intermediate selected from the group consisting of 4FBA, TPAL, pAMBA and pAMB.

12. The method of claim 10, further comprising converting the TPA to 4FBA, converting the 4FBA to TPAL, converting the TPAL to pAMB, and converting the pAMB to the pXYL.

13. The method of claim 10, further comprising converting the TPA to 4FBA, converting the 4FBA to pAMBA, converting the pAMBA to pAMB, and converting the pAMB to the pXYL.

14. The method of claim 10, wherein the pXYL has a molar yield of 30-50%.

15. The method of claim 8, wherein the PET-derived aryl carboxylic acid is mono-(2-hydroxyethyl)-terephthalic acid (MHET) and the amine product is para-(aminomethyl)benzoic acid (pAMBA).

16. The method of claim 15, further comprising producing mono-(2-hydroxyethyl)-para-(aminomethyl)benzoic acid (MHE-pAMBA) as an intermediate.

17. The method of claim 15, further comprising converting the MHET to mono-(2-hydroxyethyl)-para-(aminomethyl)benzoic acid (MHE-pAMBA), and converting the MHE-pAMBA to the pAMBA.

18. The method of claim 1, wherein the ω-TA consists of an amino acid sequence having at least 80% identity to the amino acid sequence of SEQ ID NO: 18.

19-31. (canceled)

32. A method for reducing a substrate, comprising incubating the substrate and a carboxylic acid reductase (CAR) in a reduction mixture to produce a reduction product, wherein the substrate is selected from the group consisting of terephthalic acid (TPA), 4-formylbenzoic acid (4FBA), mono-(2-hydroxyethyl)-terephthalic acid (MHET), monomethyl terephthalate (mmTPA), and para-(aminomethyl)benzoic acid (pAMBA), and wherein the CAR consists of an amino acid sequence having at least 80% identity to an amino acid sequence selected from the group consisting of SEQ ID NOs: 1-17.

33-49. (canceled)

50. A method for transferring an amine to an aldehyde, comprising incubating the aldehyde and an amine transferring enzyme in an amine transferring mixture to produce an amine product, wherein the amine transferring enzyme is selected from the group consisting of a ω-transaminase (TA), an amine transaminase and a reductive aminase.

51-61. (canceled)
